(19) United States
(12) Patent Application Publication
(10) Pub. No.: US 2021/0038677 A1
(43) Pub. Date: Feb. 11, 2021

(54) METHODS AND COMPOSITIONS FOR PREVENTING OR TREATING LEBER'S HEREDITARY OPTIC NEUROPATHY

(71) Applicant: Stealth BioTherapeutics Corp, Monaco (MC)

(72) Inventor: D. Travis Wilson, Newton, MA (US)

(73) Assignee: Stealth BioTherapeutics Corp, Monaco (MC)

(21) Appl. No.: 16/821,766

(22) Filed: Mar. 17, 2020

Related U.S. Application Data
(63) Continuation of application No. 16/179,483, filed on Nov. 2, 2018, now abandoned.
Application No. 16/179,483 is a continuation of application No. 14/908,312, filed on Jan. 28, 2016, now abandoned.
(60) Provisional application No. 61/861,244, filed on Aug. 1, 2013.

(51) Int. Cl. A61K 38/07; A61K 45/06; A61K 38/06; A61K 38/10; A61K 38/08

(52) U.S. Cl. CPC A61K 38/07; A61K 45/06; A61K 38/08; A61K 38/10; A61K 38/06

(57) ABSTRACT

The disclosure generally describes methods of preventing or treating Lebers hereditary optic neuropathy (LHON). The methods comprise administering an effective amount of an aromatic-cationic peptide to subjects in need thereof.

### CROSS-REFERENCE TO RELATED APPLICATIONS

This application claims the benefit of and priority to U.S. Application No. 61/861,244, filed Aug. 1, 2013, the content of which is incorporated herein by reference in its entirety.

### TECHNICAL FIELD

The present technology relates generally to compositions and methods of preventing or treating an ophthalmic disease. In particular, the present technology relates to methods and compositions for treating or preventing Leber's hereditary optic neuropathy (LHON).

### BACKGROUND

The following description is provided to assist the understanding of the reader. None of the information provided or references cited is admitted to be prior art to the present technology.

Leber's hereditary optic neuropathy (LHON) or Leber optic atrophy is a mitochondrially inherited degeneration of retinal ganglion cells (RGCs) and their axons that leads to an acute or subacute loss of central vision. The is maternally transmitted, as it is primarily due to mutations in the mitochondrial genome, usually point mutations in one of three subunits of complex I of the oxidative phosphorylation chain in mitochondria.

### SUMMARY

The present technology relates generally to the treatment or prevention of Leber's hereditary optic neuropathy (LHON) in mammals through administration of therapeutically effective amounts of aromatic-cationic peptides to subjects in need thereof.

In one aspect, the present disclosure provides a method of treating or preventing Leber's hereditary optic neuropathy (LHON) in a mammalian subject in need thereof, the method comprising administering to the subject a therapeutically effective amount of the peptide D-Arg-2′,6′-Dmt-Lys-Phe-NH₂, or a pharmaceutically acceptable salt thereof, such as acetate or trifluoroacetate salt.

In one aspect, the disclosure provides a method of treating or preventing Leber's hereditary optic neuropathy (LHON) in a mammalian subject, comprising administering to said mammalian subject a therapeutically effective amount of an aromatic-cationic peptide. In some embodiments, the aromatic-cationic peptide is a peptide having:

at least one net positive charge;

a minimum of four amino acids;

a maximum of about twenty amino acids;

a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1. In particular embodiments, the mammalian subject is a human.

In one embodiment, 2pₘ is the largest number that is less than or equal to r+1, and may be equal to pₜ. The aromatic-cationic peptide may be a water-soluble peptide having a minimum of two or a minimum of three positive charges.

In one embodiment, the peptide comprises one or more non-naturally occurring amino acids, for example, one or more D-amino acids. In some embodiments, the C-terminal carboxyl group of the amino acid at the C-terminus is amidated. In certain embodiments, the peptide has a minimum of four amino acids. The peptide may have a maximum of about 6, a maximum of about 9, or a maximum of about 12 amino acids.

In one embodiment, the peptide may have the formula Phe-D-Arg-Phe-Lys-NH₂ or 2′,6′-Dmp-D-Arg-Phe-Lys-NH₂. In a particular embodiment, the aromatic-cationic peptide has the formula D-Arg-2′,6′-Dmt-Lys-Phe-NH₂.

In one embodiment, the peptide is defined by formula I:

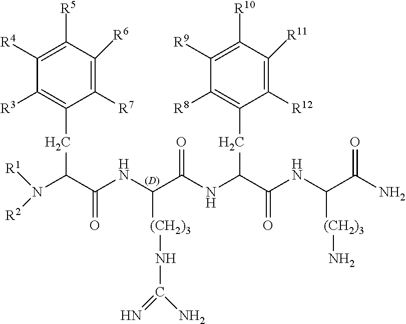

wherein R¹ and R² are each independently selected from

(i) hydrogen;

(ii) linear or branched C₁-C₆ alkyl;

(iii)

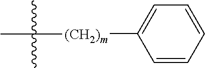

where m=1-3;

(iv)

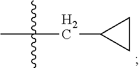 [SMILES: *CC1CC1]

(v)

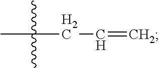 [SMILES: *CC=C]

R³, R⁴, R⁵, R⁶, R⁷, R⁸, R⁹, R¹⁰, R¹¹ and R¹² are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and n is an integer from 1 to 5.

In a particular embodiment R¹, R², R³, R⁴, R⁵, R⁶, R⁷, R⁸, R⁹, R¹⁰, R¹¹, and R¹² are all hydrogen; and n is 4. In another embodiment R¹, R², R³, R⁴, R⁵, R⁶, R⁷, R⁸, R⁹, and R¹¹ are all hydrogen; R⁸ and R¹² are methyl; R¹⁰ is hydroxyl; and n is 4.

In one embodiment, the peptide is defined by formula II:

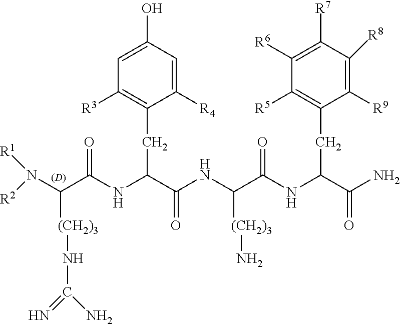

wherein R¹ and R² are each independently selected from

(i) hydrogen;

(ii) linear or branched C₁-C₆ alkyl;

(iii)

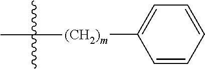

where m=1-3;

(iv)

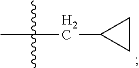 [SMILES: *CC1CC1]

(v)

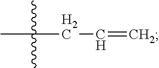 [SMILES: *CC=C]

R³ and R⁴ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo;

R⁵, R⁶, R⁷, R⁸, and R⁹ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and

n is an integer from 1 to 5.

The aromatic-cationic peptides may be administered in a variety of ways. In some embodiments, the peptides may be administered intraocularly, orally, topically, intranasally, intravenously, subcutaneously, or transdermally (e.g., by iontophoresis).

In one aspect, the present disclosure provides a pharmaceutical composition comprising a therapeutically effective amount of the peptide D-Arg-2′,6′-Dmt-Lys-Phe-NH₂ formulated for topical, iontophoretic, or intraocular administration.

In one aspect, the present disclosure provides an ophthalmic formulation comprising a therapeutically effective amount of the peptide D-Arg-2′,6′-Dmt-Lys-Phe-NH₂. In some embodiments, the formulation is soluble in the cornea, aqueous humor, and/or lens of the eye. In some embodiments, the formulation further comprises a preservative. In some embodiments, the preservative is present in a concentration of less than 1%.

In some embodiments, the formulation further comprises one or more active agents selected from the group consisting of: a vitamin, an antioxidant, a metal complexer, an anti-inflammatory drug, an antibiotic, and an antihistamine. In some embodiments, the antioxidant is vitamin A, vitamin C, vitamin E, lycopene, selenium, α-lipoic acid, coenzyme Q, glutathione, curcumin, idebenone, or a carotenoid. In some embodiments, the vitamin is selected from the group consisting of: vitamin B2 and vitamin B12.

Additionally or alternatively, in some embodiments, the formulation further comprises an active agent selected from the group consisting of: aceclidine, acetazolamide, anecortave, apraclonidine, atropine, azapcntacene, azclastinc, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

### BRIEF DESCRIPTION OF THE DRAWINGS

FIG. 1 shows the schedule of clinical parameters to be assessed at each patient visit. Vital signs include temperature, respiratory rate, sitting blood pressure and pulse. Blood and urine for safety will consist of: hematology panel, clinical chemistry panel and urinalysis. Urine pregnancy tests will be carried out on women of childbearing potential only. Manifest refraction will be conducted at Screening and Month 18 visits only. Screening procedures may be completed on more than one day, so long as all procedures are completed during the Screening Period. If Screening and Baseline visits are performed on separate days, the following tests should be repeated at Baseline: vital signs, Blood and Urine for Safety, ECG, urine pregnancy test and Humphrey Stimulus III visual field testing.

### DETAILED DESCRIPTION

It is to be appreciated that certain aspects, modes, embodiments, variations and features of the technology are described below in various levels of detail in order to provide a substantial understanding of the present technology.

It is known in the art that aromatic-cationic peptides of the present technology, such as D-Arg-2′,6′-Dmt-Lys-Phe-NH₂, possess anti-oxidant properties, including the capacity to reduce the rate of lipid oxidation, peroxidation, mitochondrial H₂O₂ production, and intracellular reactive oxygen species (ROS) production. It is further known in the art that aromatic-cationic peptides of the present technology, such as D-Arg-2′,6′-Dmt-Lys-Phe-NH₂, localize to the mitochondria, and have the capacity to inhibit caspase activation and apoptosis. These and other properties of aromatic-cationic peptides of the present technology, such as D-Arg-2′,6′-Dmt-Lys-Phe-NH₂, are demonstrated in U.S. application Ser. No. 11/040,242 (U.S. Pat. No. 7,550,439) and Ser. No. 10/771,232 (U.S. Pat. No. 7,576,061). Accordingly, aromatic-cationic peptides of the present technology, such as D-Arg-2′,6′-Dmt-Lys-Phe-NH₂, are useful in the prevention and treatment of diseases and conditions caused by, resulting from, or otherwise associated with such cellular events, such as Leber's hereditary optic neuropathy (LHON).

Leber's hereditary optic neuropathy (LHON) is a maternally inherited blinding disease with variable penetrance. LHON is usually due to one of three pathogenic mitochondrial DNA (mtDNA) point mutations. These mutations are at nucleotide positions 11778 G to A, 3460 G to A and 14484 T to C, respectively in the ND4, ND1 and ND6 subunitgenes of complex I of the oxidative phosphorylation chain in mitochondria. Reduced efficiency of ATP synthesis and increased oxidative stress are believed to sensitize the retinal ganglion cells to apoptosis. Different therapeutic strategies are considered to counteract this pathogenic mechanism. However, potential treatments for the visual loss are complicated by the fact that patients are unlikely to benefit after optic atrophy occurs. There is no proven therapy to prevent or reverse the optic neuropathy in LHON. Results from a recent trial with idebenone hold promise to limit neurodegeneration and improve final outcome, promoting recovery of visual acuity. Other therapeutic options are under scrutiny, including gene therapy, agents increasing mitochondrial biogenesis, and anti-apoptotic drugs.

Leber's hereditary optic neuropathy (LHON) is a maternally inherited disease characterized by severe visual loss, which usually does not manifest until young adulthood. Maternal transmission is due to a mitochondrial DNA (mtDNA) mutation affecting nucleotide positions (nps) 11778/ND4, 14484/ND6, or 3460/ND1. These three mutations, affecting respiratory complex I, account for about 95% of LHON cases. Patients inherit multicopy mtDNA entirely from the mother (via the oocyte). The mitochondria may carry only wild-type or only LHON mutant mtDNA (homoplasmy), or a mixture of mutant and wild-type mtDNA (heteroplasmy). Only high loads of mutant heteroplasmy or, most frequently, homoplasmic mutant mtDNA in the target tissue put the subject at risk for blindness from LHON.

Except for patients carrying the 14484/ND6 mutation (who present with a more benign disease course), most patients remain legally blind. Typically, a subject in his second or third decade of life will present with abrupt and profound loss of vision in one eye, followed weeks to months later by similar loss of vision in the other eye. LHON may occur later in life and affects both men and women. Environmental factors may trigger the visual loss but do not fully explain why only certain individuals within a family become symptomatic.

### LHON Epidemiology

LHON is one of the most frequently occurring mitochondrial diseases. The prevalence of visual loss from LHON has been reported to be approximately 1 in 30,000 in Northeast England, 1 in 40,000 in The Netherlands, and 1 in 50,000 in Finland. However, the disease remains underestimated: many patients are not adequately diagnosed or are given an inadequate description of optic atrophy, and many are simply misdiagnosed. Furthermore, most individuals carrying the LHON mutation remain unaffected, though a subset of them may develop the disease later in life. The minimum prevalence for the LHON mtDNA mutations is probably about 15 per 100,000, which is similar to many autosomal inherited neurologic diseases.

Penetrance for the disease (percent affected of total number of mutation carriers) is much higher for men than for women. For example, in a well-studied, very large Brazilian 11778/ND4 pedigree, about 45% of the males and 10% of the females lost vision. Penetrance also varies greatly between families and even within the same pedigree. Factors that affect penetrance may include heteroplasmy, environmental factors, and the mitochondrial DNA background, as well as nuclear modifying genes. It is for this last reason that the likelihood of visual loss has been reported to be greater if the mother is affected, even within the same pedigree.

### LHON Pathophysiology

The primary etiologic cause of LHON is an mtDNA mutation, which is a necessary determinant but not sufficient to lead to visual loss. In fact, most individuals carrying one of the three mtDNA mutations remain asymptomatic, even though they may show subclinical changes such as retinal nerve fiber layer (RNFL) thickening on optical coherence tomography (OCT) or subtle dyschromatopsia. A subgroup of these unaffected mutation carriers may convert and become affected, suffering an abrupt and serious loss of central vision.

All three LHON mutations affect different subunits of complex I, the first site of the mitochondrial electron transport chain. Complex I dysfunction due to the LHON mutations may lead to a combination of reduced adenosine triphosphate (ATP) synthesis, increased oxidative stress, and predisposition for cells to undergo apoptosis. The severity of the biochemical phenotype is higher for the 3460/ND1 and the 11778/ND4 mutations and milder for the 14484/ND6 mutation.

The mechanism by which LHON mutations result in the selective death of retinal ganglion cells (RGCs) is unclear. However, it is widely accepted that RGC death is the result of bioenergetic defects, chronic oxidative stress, or a combination of both. It is thought that these mechanisms lead to changes in mitochondrial membrane potential, lowering the threshold for the mitochondrial permeability transition pore (MPTP) opening, and initiating mitochondrially driven apoptosis.

Histopathologic descriptions of molecularly characterized LHON patients have demonstrated a dramatic loss of RGCs and their axons, which constitute the nerve fiber layer and optic nerve. The centrally located, small-caliber fibers of the papillomacular bundle (PMB) were most damaged, and the larger axons on the periphery were most spared. Mitochondria accumulate in the RNFL, especially in the unmyelinated portion anterior to the lamina cribrosa, as this is the area with the greatest energy requirements. The particularly high energy demands of the unmyelinated RNFL may explain why the optic nerve, which represents the coalescence of these fibers as they course towards the brain, is the target tissue in LHON.

### LHON Clinical Presentation

The patient classically presents with painless, subacute loss of vision in one eye. The visual acuity is usually worse than 20/400, and there is optic nerve dysfunction manifested as large and dense central or cecocentral scotomas on visual fields. Fundus examination in LHON may show telangiectatic capillaries and pseudoedema of the optic disc with surrounding swelling of the RNFL. Over time, there is loss of the PMB with corresponding atrophy of the temporal optic nerve, which eventually will extend to the other quadrants, leading to diffuse optic atrophy. The visual loss in LHON is usually permanent, although a subgroup of patients may spontaneously recover some visual acuity. This recovery is particularly frequent with the 14484/ND6 mutation. One remarkable aspect of LHON is the tissue specificity. The optic nerve is singularly involved, with preferential loss of the smallest fibers that constitute the PMB. Loss of vision is usually the only clinical manifestation, notwithstanding reports of patients with cardiac, skeletal, or neurologic dysfunction.

### LHON Differential Diagnosis

LHON patients present with subacute visual loss and optic neuropathy. Fundus examination will usually rule out any retinopathy. Hence, the differential diagnosis begins with the optic neuropathies. Usually, the subacute tempo of the visual loss is very helpful. Compressive lesions involving the optic nerve have a slowly progressive course. So too does chronic papilledema from brain tumors or idiopathic intracranial hypertension (pseudotumor cerebri). Glaucoma also is a much slower and progressive process and the optic disc cupping is usually obvious. Ischemic optic neuropathies produce a very abrupt loss of vision, but the optic disc appearance, including peripapillary hemorrhages, is distinctive. Hence, a young adult with painless subacute visual loss is likely to have an inflammatory or infiltrative optic neuropathy. These etiologies are revealed by fundus examination and neuroimaging. An infiltrative optic neuropathy is usually evident by the thickened appearance of the optic disc and by the leakage of dye during fluorescein angiography. MRI studies of the brain help reveal any infiltrative or inflammatory lesions of the optic nerve, or lesions elsewhere, as in multiple sclerosis.

However, as in many neuro-ophthalmologic diseases, the most revealing part of the examination comes from the history. In addition to the tempo of visual loss, the patient with LHON can often provide a history of visual loss in family members along the maternal line. The history will also confirm the absence of other systemic or constitutional symptoms. After the patient has lost vision in the second eye, the diagnosis becomes much easier. In addition to all the points above, the features of both eyes can now be compared. Bilaterally symmetric optic neuropathies are almost always due to mitochondrial disease. This becomes even more certain with bilaterally symmetrical central or cecocentral scotomas on visual field testing. Mitochondrial optic neuropathies fall into three categories: 1) LHON, 2) dominant optic atrophy (DOA), and 3) nutritional and toxic optic neuropathies. The disease segregation in DOA will involve paternal as well as maternal transmission. Furthermore, the visual loss occurs at a younger age (usually before age 10) and progresses slowly over many years, often leveling off at 20/100 or 20/200. This is easily distinguishable from LHON.

Nutritional and toxic etiologies must also be investigated by a careful history. Folate and vitamin B deficiencies are usually associated with a very poor diet over a long course. There may also be an associated anemia. Toxic agents that can produce a mitochondrial optic neuropathy include several antibiotics.

### LHON Diagnostic Testing

LHON can usually be diagnosed clinically. Confirmation can be made by blood testing of the mtDNA to reveal one of the three common mutations. Even if this test is negative, however, LHON may still be considered, as about 5% of cases are not due to the three common LHON mutations. Complete mtDNA sequence analysis may be recommended if the clinical diagnosis of LHON remains as a strong indication, or if there is evidence of maternal transmission of blindness. DNA testing of primary LHON mutations is especially useful in atypical presentations or in the absence of a clear family history of LHON or optic atrophy of unknown etiology limited to the maternal side of the pedigree. Ophthalmologic and psychophysical tests are also useful. In LHON, there is absence of dye leakage at the optic disc on fluorescein angiography. In the acute phase of the disease, OCT demonstrates thickening of the RNFL around the optic nerve; on subsequent examinations, it reveals thinning of the RNFL.

Unaffected mutation carriers may show subclinical abnormalities. Examination and testing of 75 asymptomatic carriers in a large Brazilian family with the 11778/ND4 mutation revealed microangiopathy and swelling of the RNFL in about 15% of the eyes. These mutation carriers also exhibited corresponding relative central visual field defects on Humphrey visual field tests. Furthermore, they often showed subtle deficits in color vision and contrast sensitivity, as well as thickening of the RNFL on OCT testing.

### LHON Risk Factors

Environmental risk factors may be important triggers of the conversion to active LHON in unaffected carriers. One study of a large Brazilian LHON pedigree (332 individuals, 97 on the maternal line, all carrying a homoplasmic 11778/ND4 mutation and J-haplogroup) showed a doubling of disease risk with high consumption of either alcohol or tobacco. A subsequent multicenter survey of a cohort of 402 LHON patients, carrying the three primary mutations, also found a significant role in disease risk for tobacco, in particular, and alcohol use. Smoke in general (not just tobacco smoking) may also trigger LHON, as some reported cases have been associated with exposure to smoke from tire fires or malfunctioning stoves. Further triggers of LHON may be antibiotics such as ethambutol, chloramphenicol, linezolid, aminoglycosides, and antiretroviral drugs (for HIV). All of these are known for interfering with mitochondrial respiratory function.

Agents that may prompt the conversion in Leber's hereditary optic neuropathy include, but are not limited to, for example, antibiotics, ethambutol, aminoglycosides, chloramphenicol, linezolid, Zidovudine (AZT) and other antiretroviral drugs, toxins, smoke (including tobacco), ethanol, pesticides, cyanide, and methanol.

### LHON Treatment

Most treatment options in LHON target excessive production of reactive oxygen species. Antioxidants such as glutathione, Trolox (a derivative of vitamin E), and coenzyme Q-10 have demonstrated modest protective effects in vitro. A current clinical trial in Thailand is investigating the efficacy of curcumin, another compound with antioxidant properties, in treating LHON patients.

Coenzyme Q10 is a mitochondrial cofactor that shuttles electrons from complexes I and II to complex III. Coenzyme Q10 (or ubiquinone) is available as a nutritional supplement. A few case reports of treatment with coenzyme Q10 have been published, but the lack of any successful case series gives rise to skepticism about this treatment. One likely limitation of treatment with exogenous coenzyme Q10 relates to its poor delivery crossing lipid membranes to mitochondria

Idebenone, a coenzyme Q10 derivative, is reported to have higher delivery to mitochondria as well as a higher efficiency in crossing the blood-brain barrier. Successful treatment with idebenone has been described in a few case reports and retrospective case series. One such study evaluated the treatment of 28 Japanese patients with LHON who carried all three mutations. The authors divided these patients into two groups: an untreated group and a group treated with a combination of idebenone, riboflavin (vitamin B2), and ascorbic acid (vitamin C). The two cohorts of LHON patients had an equal distribution of mtDNA mutation types. The visual recovery was significantly earlier for treated patients carrying the 11778/ND4 mutation and was limited to small openings that appeared in the paracentral visual field (fenestrations).

In a recently reported study, seven LHON patients treated with idebenone alone (about 450 mg/d) showed recovery of visual acuity, color vision, and visual fields. One 11778/ND4 LHON patient improved from counting-fingers vision in both eyes to visual acuities of 20/20 and 20/30 with associated shrinkage of the central scotomas from a diameter of about 20 degrees to less than 5 degrees.

Also recently, the Rescue of Hereditary Optic Disease Outpatient Study (RHODOS) was concluded. In this large, double-blind, randomized, placebo-controlled clinical trial in a series of 85 LHON patients, treated patients were given idebenone (900 mg/d) for 24 weeks. The preliminary press release highlighted that patients taking idebenone had better final visual acuity than the placebo group.

Topical brimonidine, an alpha-2 agonist, vitamins (especially folic acid and vitamins C, E, B2, and B12), and nutritional supplements have also been used for the treatment or prevention of LHON.

Other strategies proposed to bypass the complex I dysfunction in LHON are based on a gene-therapy approach. However, none of these approaches are currently used in patients; they remain experimental pending further evidence of their safety and usefulness.

LHON is due to mutations affecting the mtDNA-encoded subunits of complex I (11778/ND4, 3460/ND1, 14484ND4). One strategy of gene therapy is the so-called nuclear allotopic expression of a mitochondrial gene. Briefly, in order to express a wild-type version of the mtDNA encoded ND subunits in the nucleus, they first need to be recoded according to the slightly different coding system of nuclear DNA. Then, the recoded wild-type ND subunit is engineered to carry the mitochondrial import signal and is delivered by an AAV vector to the nucleus of the target cells (RGCs). Thus, the nuclear-encoded wild-type ND subunit will be expressed in the cell cytoplasm and transported to mitochondria, where it is assumed to co-assemble in complex 1. This wild-type ND subunit will be competing with the mitochondrial-encoded mutant ND subunit, thus potentially complementing the biochemical defect. However, serious doubts have been cast on this approach recently, and caution must be exercised before the stage of clinical trials in patients is reached.

Another strategy is based on the xenotopic expression of an alternative oxidase, such as the Saccharomyces cerevisiae single subunit NADH oxidase Ndi1, in mammalian cells. This can re-establish the electron flow to coenzyme Q bypassing the complex I defect, but without coupled proton translocation, thus missing the energy-conserving function of complex I. By this means, the downstream respiratory chain is fed again with the electron flow, re-establishing a sufficiently efficient oxidative phosphorylation. This gene therapy approach has been successfully tried in an experimental animal model mimicking LHON.

Other therapeutic strategies are proposed to provide a compensatory mechanism to prevent the loss of vision in unaffected individuals carrying the mutation, and to inhibit the apoptotic program in RGCs once the acute phase has started.

The compensatory mechanism is based on activating mitochondrial biogenesis. To this end, drugs such as bezafibrate and rosiglitazone are being tested in vitro; they act as peroxisome proliferator-activated receptor γ (PPARγ) activators and, through PPARγ coactivator α (PGC1α), enhance mitochondrial biogenesis. A similar result may be achieved by estrogens or estrogen-related compounds, which recently have been shown to activate mitochondrial gene expression, including antioxidant enzymes, and to increase mtDNA copy number.

A class of drugs that includes as a prototypic example cyclosporine A can abort the apoptotic program by holding closed the MPTP. These drugs may be beneficial in the very early stages of LHON by modifying the natural disease progression.

### General

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectorsfor Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent, drug, or peptide to a subject includes any route of introducing or delivering to a subject a compound to perform its intended function. Administration can be carried out by any suitable route, including orally, intraocularly, intranasally, parenterally (intravenously, intramuscularly, intraperitoneally, or subcutaneously), or topically. Administration includes self-administration and the administration by another.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “effective amount” refers to a quantity sufficient to achieve a desired therapeutic and/or prophylactic effect, e.g., an amount which results in the prevention of, or a decrease in, the symptoms associated with an ophthalmic condition, such as Leber's hereditary optic neuropathy (LHON). The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In the methods described herein, the aromatic-cationic peptides may be administered to a subject having one or more signs or symptoms of an ophthalmic condition such as Leber's hereditary optic neuropathy (LHON). For example, a “therapeutically effective amount” of the aromatic-cationic peptides is meant levels in which the physiological effects of an ophthalmic condition such as Leber's hereditary optic neuropathy (LHON) are, at a minimum, ameliorated.

An “isolated” or “purified” polypeptide or peptide is substantially free of cellular material or other contaminating polypeptides from the cell or tissue source from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include enzymes, hormones and other proteinaceous and nonproteinaceous solutes.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the terms “treating” or “treatment” or “alleviation” refers to both therapeutic treatment and prophylactic measures, wherein the object is to slow down (lessen) the targeted pathologic condition or disorder. A subject is successfully “treated” for an ophthalmic condition if, after receiving a therapeutic amount of the aromatic-cationic peptides according to the methods described herein, the subject shows observable and/or measurable reduction in or absence of one or more signs and symptoms of an ophthalmic condition. It is also to be appreciated that the various modes of treatment or prevention of medical conditions as described are intended to mean “substantial”, which includes total but also less than total treatment, and wherein some biologically or medically relevant result is achieved.

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, “Leber's hereditary optic neuropathy (LHON)” or “Leber optic atrophy” refer to a mitochondrially inherited disorder that results in degeneration of retinal ganglion cells. As used herein, the term encompasses neuropathies caused by mutations in mitochondrial DNA (mtDNA), including, but not limited to, for example, point mutations in genes encoding the ND4, ND1 and ND6 subunits of complex I of the oxidative phosphorylation chain. Such mutations include, but are not limited to, for example, 11778 G to A, 3460 G to A, and 14484 T to C of the ND4, ND1 and ND6 subunit sequences, respectively.

### Aromatic-Cationic Peptides

The present technology relates to the treatment or prevention Leber's hereditary optic neuropathy (LHON) by administration of aromatic-cationic peptides of the present technology. Without wishing to be limited by theory, the aromatic-cationic peptides may treat or prevent LHON by reducing the severity or occurrence of oxidative damage in the eye. It is expected that administration of aromatic-cationic peptides will not only be effective for the treatment or prevention of LHON, but that administration of the peptides in combination with additional therapeutic agents will have synergistic effects in treatment or prevention of the disease. For example, administration of the peptides in combination conventional or newly developed agents for the treatment of LHON will exhibit synergistic effects.

The aromatic-cationic peptides of the present technology are water-soluble and highly polar. Despite these properties, the peptides can readily penetrate cell membranes. The aromatic-cationic peptides typically include a minimum of three amino acids or a minimum of four amino acids, covalently joined by peptide bonds. The maximum number of amino acids present in the aromatic-cationic peptides is about twenty amino acids covalently joined by peptide bonds. Suitably, the maximum number of amino acids is about twelve, about nine, or about six.

The amino acids of the aromatic-cationic peptides can be any amino acid. As used herein, the term “amino acid” is used to refer to any organic molecule that contains at least one amino group and at least one carboxyl group. Typically, at least one amino group is at the a position relative to a carboxyl group. The amino acids may be naturally occurring. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Other naturally occurring amino acids include, for example, amino acids that are synthesized in metabolic processes not associated with protein synthesis. For example, the amino acids ornithine and citrulline are synthesized in mammalian metabolism during the production of urea. Another example of a naturally occurring amino acid include hydroxyproline (Hyp).

The peptides optionally contain one or more non-naturally occurring amino acids. Suitably, the peptide has no amino acids that are naturally occurring. The non-naturally occurring amino acids may be levorotary (L-), dextrorotatory (D-), or mixtures thereof. Non-naturally occurring amino acids are those amino acids that typically are not synthesized in normal metabolic processes in living organisms, and do not naturally occur in proteins. In addition, the non-naturally occurring amino acids suitably are also not recognized by common proteases. The non-naturally occurring amino acid can be present at any position in the peptide. For example, the non-naturally occurring amino acid can be at the N-terminus, the C-terminus, or at any position between the N-terminus and the C-terminus.

The non-natural amino acids may, for example, comprise alkyl, aryl, or alkylaryl groups not found in natural amino acids. Some examples of non-natural alkyl amino acids include α-aminobutyric acid, β-aminobutyric acid, γ-aminobutyric acid, δ-aminovaleric acid, and ε-aminocaproic acid. Some examples of non-natural aryl amino acids include ortho-, meta, and para-aminobenzoic acid. Some examples of non-natural alkylaryl amino acids include ortho-, meta-, and para-aminophenylacetic acid, and γ-phenyl-β-aminobutyric acid. Non-naturally occurring amino acids include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva) and norleucine (Nle).

Another example of a modification of an amino acid in a peptide is the derivatization of a carboxyl group of an aspartic acid or a glutamic acid residue of the peptide. One example of derivatization is amidation with ammonia or with a primary or secondary amine, e.g. methylamine, ethylamine, dimethylamine or diethylamine. Another example of derivatization includes esterification with, for example, methyl or ethyl alcohol. Another such modification includes derivatization of an amino group of a lysine, arginine, or histidine residue. For example, such amino groups can be acylated. Some suitable acyl groups include, for example, a benzoyl group or an alkanoyl group comprising any of the C₁-C₄ alkyl groups mentioned above, such as an acetyl or propionyl group.

The non-naturally occurring amino acids may be resistant or insensitive to common proteases. Examples of non-naturally occurring amino acids that are resistant or insensitive to proteases include the dextrorotatory (D-) form of any of the above-mentioned naturally occurring L-amino acids, as well as L- and/or D-non-naturally occurring amino acids. The D-amino acids do not normally occur in proteins, although they are found in certain peptide antibiotics that are synthesized by means other than the normal ribosomal protein synthetic machinery of the cell. As used herein, the D-amino acids are considered to be non-naturally occurring amino acids.

In order to minimize protease sensitivity, the peptides should have less than five, less than four, less than three, or less than two contiguous L-amino acids recognized by common proteases, irrespective of whether the amino acids are naturally or non-naturally occurring. Suitably, the peptide has only D-amino acids, and no L-amino acids. If the peptide contains protease sensitive sequences of amino acids, at least one of the amino acids is a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine.

The aromatic-cationic peptides should have a minimum number of net positive charges at physiological pH in comparison to the total number of amino acid residues in the peptide. The minimum number of net positive charges at physiological pH will be referred to below as (pₘ). The total number of amino acid residues in the peptide will be referred to below as (r). The minimum number of net positive charges discussed below are all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

“Net charge” as used herein refers to the balance of the number of positive charges and the number of negative charges carried by the amino acids present in the peptide. In this specification, it is understood that net charges are measured at physiological pH. The naturally occurring amino acids that are positively charged at physiological pH include L-lysine, L-arginine, and L-histidine. The naturally occurring amino acids that are negatively charged at physiological pH include L-aspartic acid and L-glutamic acid.

Typically, a peptide has a positively charged N-terminal amino group and a negatively charged C-terminal carboxyl group. The charges cancel each other out at physiological pH. As an example of calculating net charge, the peptide Tyr-Arg-Phe-Lys-Glu-His-Trp-D-Arg has one negatively charged amino acid (i.e., Glu) and four positively charged amino acids (i.e., two Arg residues, one Lys, and one His). Therefore, the above peptide has a net positive charge of three.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≤ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≤ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, a minimum of two net positive charges, or a minimum of three net positive charges.

It is also important that the aromatic-cationic peptides have a minimum number of aromatic groups in comparison to the total number of net positive charges (pₜ). The minimum number of aromatic groups will be referred to below as (a). Naturally occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≤ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≤ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pₜ) are equal.

Carboxyl groups, especially the terminal carboxyl group of a C-terminal amino acid, may be amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethylamido, N-methyl-N-ethylamido, N-phenylamido or N-phenyl-N-ethylamido group. The free carboxylate groups of the asparagine, glutamine, aspartic acid, and glutamic acid residues not occurring at the C-terminus of the aromatic-cationic peptides may also be amidated wherever they occur within the peptide. The amidation at these internal positions may be with ammonia or any of the primary or secondary amines described above.

In one embodiment, the aromatic-cationic peptide is a tripeptide having two net positive charges and at least one aromatic amino acid. In a particular embodiment, the aromatic-cationic peptide is a tripeptide having two net positive charges and two aromatic amino acids.

Aromatic-cationic peptides include, but are not limited to, the following peptide examples:

Lys-D-Arg-Tyr-NH₂
Phe-D-Arg-His
D-Tyr-Trp-Lys-NH₂
Trp-D-Lys-Tyr-Arg-NH₂
Tyr-His-D-Gly-Met
Phe-Arg-D-His-Asp
Tyr-D-Arg-Phe-Lys-Glu-NH₂
Met-Tyr-D-Lys-Phe-Arg
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-Tyr-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-His-D-Lys-Arg-Trp-
NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-
NH₂
Phe-Try-Lys-D-Arg-Trp-His-D-Lys-D-Lys-Glu-Arg-D-
Tyr-Thr
Tyr-Asp-D-Lys-Tyr-Phe-D-Lys-D-Arg-Phe-Pro-D-Tyr-
His-Lys
Glu-Arg-D-Lys-Tyr-D-Val-Phe-D-His-Trp-Arg-D-Gly-
Tyr-Arg-D-Met-NH₂
Arg-D-Leu-D-Tyr-Phe-Lys-Glu-D-Lys-Arg-D-Trp-Lys-D-
Phe-Tyr-D-Arg-Gly
D-Glu-Asp-Lys-D-Arg-D-His-Phe-Phe-D-Val-Tyr-Arg-
Tyr-D-Tyr-Arg-His-Phe-NH₂
Asp-Arg-D-Phe-Cys-Phe-D-Arg-D-Lys-Tyr-Arg-D-Tyr-
Trp-D-His-Tyr-D-Phe-Lys-Phe
His-Tyr-D-Arg-Trp-Lys-Phe-D-Asp-Ala-Arg-Cys-D-Tyr-
His-Phe-D-Lys-Tyr-His-Ser-NH₂
Gly-Ala-Lys-Phe-D-Lys-Glu-Arg-Tyr-His-D-Arg-D-Arg-
Asp-Tyr-Trp-D-His-Trp-His-D-Lys-Asp
Thr-Tyr-Arg-D-Lys-Trp-Tyr-Glu-Asp-D-Lys-D-Arg-His-
Phe-D-Tyr-Gly-Val-Ile-D-His-Arg-Tyr-Lys-NH₂

In one embodiment, a peptide that has mu-opioid receptor agonist activity has the formula Tyr-D-Arg-Phe-Lys-NH₂. Tyr-D-Arg-Phe-Lys-NH₂ has a net positive charge of three, contributed by the amino acids tyrosine, arginine, and lysine and has two aromatic groups contributed by the amino acids phenylalanine and tyrosine. The tyrosine of Tyr-D-Arg-Phe-Lys-NH₂ can be a modified derivative of tyrosine such as in 2′,6′-dimethyltyrosine (2′,6′-Dmt) to produce the compound having the formula 2′,6′-Dmt-D-Arg-Phe-Lys-NH₂. 2′,6′-Dmt-D-Arg-Phe-Lys-NH₂ has a molecular weight of 640 and carries a net three positive charge at physiological pH. 2′,6′-Dmt-D-Arg-Phe-Lys-NH₂ readily penetrates the plasma membrane of several mammalian cell types in an energy-independent manner (Zhao et al., J. Pharmacol Exp Ther. 304: 425-432, 2003).

Peptides that do not have mu-opioid receptor agonist activity generally do not have a tyrosine residue or a derivative of tyrosine at the N-terminus (i.e., amino acid position 1). The amino acid at the N-terminus can be any naturally occurring or non-naturally occurring amino acid other than tyrosine. In one embodiment, the amino acid at the N-terminus is phenylalanine or its derivative. Exemplary derivatives of phenylalanine include 2′-methylphenylalanine (Mmp), 2′,6′-dimethylphenylalanine (2′,6′-Dmp), N,2′,6′-trimethylphenylalanine (Tmp), and 2′-hydroxy-6′-methylphenylalanine (Hmp).

An example of an aromatic-cationic peptide that does not have mu-opioid receptor agonist activity has the formula Phe-D-Arg-Phe-Lys-NH₂. Alternatively, the N-terminal phenylalanine can be a derivative of phenylalanine such as 2′,6′-dimethylphenylalanine (2′,6′-Dmp). A variant of Phe-D-Arg-Phe-Lys-NH₂ containing 2′,6′-dimethylphenylalanine at amino acid position 1 has the formula 2′,6′-Dmp-D-Arg-Phe-Lys-NH₂. In one embodiment, the amino acid sequence of 2′,6′-Dmt-D-Arg-Phe-Lys-NH₂ is rearranged such that Dint is not at the N-terminus. An example of such an aromatic-cationic peptide that does not have mu-opioid receptor agonist activity has the formula D-Arg-2′,6′-Dmt-Lys-Phe-NH₂.

Aromatic-cationic peptides and their derivatives can further include functional analogs. A peptide is considered a functional analog of if the analog has the same function as the aromatic-cationic peptide. The analog may, for example, be a substitution variant D-Arg-2′,6′-Dmt-Lys-Phe-NH₂, wherein one or more amino acids are substituted by another amino acid.

Suitable substitution variants of aromatic-cationic peptides include conservative amino acid substitutions. Amino acids may be grouped according to their physicochemical characteristics as follows:

(a) Non-polar amino acids: Ala(A) Ser(S) Thr(T) Pro(P) Gly(G) Cys (C);

(b) Acidic amino acids: Asn(N) Asp(D) Glu(E) Gln(Q);

(c) Basic amino acids: His(H) Arg(R) Lys(K);

(d) Hydrophobic amino acids: Met(M) Leu(L) Ile(I) Val(V); and

(e) Aromatic amino acids: Phe(F) Tyr(Y) Trp(W) His (H).

Substitutions of an amino acid in a peptide by another amino acid in the same group is referred to as a conservative substitution and may preserve the physicochemical characteristics of the original peptide. In contrast, substitutions of an amino acid in a peptide by another amino acid in a different group is generally more likely to alter the characteristics of the original peptide.

In some embodiments, the aromatic-cationic peptide has a formula as shown in Table 5.

TABLE 5
Peptide Analogs with Mu-Opioid Activity

| Amino Acid | Amino Acid | Amino Acid | Amino Acid | C-Terminal |
|---|---|---|---|---|
| Position 1 | Position 2 | Position 3 | Position 4 | Modification |
| Tyr | D-Arg | Phe | Lys | NH₂ |
| Tyr | D-Arg | Phe | Orn | NH₂ |
| Tyr | D-Arg | Phe | Dab | NH₂ |
| Tyr | D-Arg | Phe | Dap | NH₂ |
| 2′,6′-Dmt | D-Arg | Phe | Lys | NH₂ |
| 2′,6′-Dmt | D-Arg | Phe | Lys-NH(CH₂)₂—NH-dns | NH₂ |
| 2′,6′-Dmt | D-Arg | Phe | Lys-NH(CH₂)₂—NH-atn | NH₂ |
| 2′,6′-Dmt | D-Arg | Phe | dnsLys | NH₂ |
| 2′,6′-Dmt | D-Cit | Phe | Lys | NH₂ |
| 2′,6′-Dmt | D-Cit | Phe | Ahp | NH₂ |
| 2′,6′-Dmt | D-Arg | Phe | Orn | NH₂ |
| 2′,6′-Dmt | D-Arg | Phe | Dab | NH₂ |
| 2′,6′-Dmt | D-Arg | Phe | Dap | NH₂ |
| 2′,6′-Dmt | D-Arg | Phe | Ahp(2-aminoheptanoic acid) | NH₂ |
| Bio-2′,6′-Dmt | D-Arg | Phe | Lys | NH₂ |
| 3′,5′-Dmt | D-Arg | Phe | Lys | NH₂ |
| 3′,5′-Dmt | D-Arg | Phe | Orn | NH₂ |
| 3′,5′-Dmt | D-Arg | Phe | Dab | NH₂ |
| 3′,5′-Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′,6′-Dmt | D-Arg | Tyr | Lys | NH₂ |
| 2′,6′-Dmt | D-Arg | Tyr | Orn | NH₂ |
| 2′,6′-Dmt | D-Arg | Tyr | Dab | NH₂ |
| 2′,6′-Dmt | D-Arg | Tyr | Dap | NH₂ |
| 2′,6′-Dmt | D-Arg | 2′,6′-Dmt | Lys | NH₂ |
| 2′,6′-Dmt | D-Arg | 2′,6′-Dmt | Orn | NH₂ |
| 2′,6′-Dmt | D-Arg | 2′,6′-Dmt | Dab | NH₂ |
| 2′,6′-Dmt | D-Arg | 2′,6′-Dmt | Dap | NH₂ |
| 3′,5′-Dmt | D-Arg | 3′,5′-Dmt | Arg | NH₂ |
| 3′,5′-Dmt | D-Arg | 3′,5′-Dmt | Lys | NH₂ |
| 3′,5′-Dmt | D-Arg | 3′,5′-Dmt | Orn | NH₂ |
| 3′,5′-Dmt | D-Arg | 3′,5′-Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′,6′-Dmt | D-Lys | Phe | Dab | NH₂ |
| 2′,6′-Dmt | D-Lys | Phe | Dap | NH₂ |
| 2′,6′-Dmt | D-Lys | Phe | Arg | NH₂ |
| 2′,6′-Dmt | D-Lys | Phe | Lys | NH₂ |
| 3′,5′-Dmt | D-Lys | Phe | Orn | NH₂ |
| 3′,5′-Dmt | D-Lys | Phe | Dab | NH₂ |
| 3′,5′-Dmt | D-Lys | Phe | Dap | NH₂ |
| 3′,5′-Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′,6′-Dmt | D-Lys | Tyr | Lys | NH₂ |
| 2′,6′-Dmt | D-Lys | Tyr | Orn | NH₂ |
| 2′,6′-Dmt | D-Lys | Tyr | Dab | NH₂ |
| 2′,6′-Dmt | D-Lys | Tyr | Dap | NH₂ |
| 2′,6′-Dmt | D-Lys | 2′,6′-Dmt | Lys | NH₂ |
| 2′,6′-Dmt | D-Lys | 2′,6′-Dmt | Orn | NH₂ |
| 2′,6′-Dmt | D-Lys | 2′,6′-Dmt | Dab | NH₂ |
| 2′,6′-Dmt | D-Lys | 2′,6′-Dmt | Dap | NH₂ |
| 2′,6′-Dmt | D-Arg | Phe | dnsDap | NH₂ |
| 2′,6′-Dmt | D-Arg | Phe | atnDap | NH₂ |
| 3′,5′-Dmt | D-Lys | 3′,5′-Dmt | Lys | NH₂ |
| 3′,5′-Dmt | D-Lys | 3′,5′-Dmt | Orn | NH₂ |
| 3′,5′-Dmt | D-Lys | 3′,5′-Dmt | Dab | NH₂ |
| 3′,5′-Dmt | D-Lys | 3′,5′-Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′,6′-Dmt | D-Arg | Phe | Arg | NH₂ |
| 2′,6′-Dmt | D-Lys | Phe | Arg | NH₂ |
| 2′,6′-Dmt | D-Orn | Phe | Arg | NH₂ |
| 2′,6′-Dmt | D-Dab | Phe | Arg | NH₂ |
| 3′,5′-Dmt | D-Dap | Phe | Arg | NH₂ |
| 3′,5′-Dmt | D-Arg | Phe | Arg | NH₂ |
| 3′,5′-Dmt | D-Lys | Phe | Arg | NH₂ |
| 3′,5′-Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′,6′-Dmt | D-Arg | 2′,6′-Dmt | Arg | NH₂ |
| 2′,6′-Dmt | D-Lys | 2′,6′-Dmt | Arg | NH₂ |
| 2′,6′-Dmt | D-Orn | 2′,6′-Dmt | Arg | NH₂ |
| 2′,6′-Dmt | D-Dab | 2′,6′-Dmt | Arg | NH₂ |
| 3′,5′-Dmt | D-Dap | 3′,5′-Dmt | Arg | NH₂ |
| 3′,5′-Dmt | D-Arg | 3′,5′-Dmt | Arg | NH₂ |
| 3′,5′-Dmt | D-Lys | 3′,5′-Dmt | Arg | NH₂ |
| 3′,5′-Dmt | D-Orn | 3′,5′-Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |

Dab = diaminobutyric
Dap = diaminopropionic acid
Dmt = dimethyltyrosine
Mmt = 2′-methyltyrosine
Tmt = N, 2′,6′-trimethyltyrosine
Hmt = 2′-hydroxy,6′-methyltyrosine
dnsDap = β-dansyl-L-α,β-diaminopropionic acid
atnDap = β-anthraniloyl-L-α,β-diaminopropionic acid
Bio = biotin

Examples of other aromatic-cationic peptides that do not activate mu-opioid receptors include, but are not limited to, the aromatic-cationic peptides shown in Table 6.

TABLE 6
Peptide Analogs Lacking Mu-Opioid Activity

| Amino Acid | Amino Acid | Amino Acid | Amino Acid | C-Terminal |
|---|---|---|---|---|
| Position 1 | Position 2 | Position 3 | Position 4 | Modification |
| D-Arg | Dmt | Lys | Phe | NH₂ |
| D-Arg | Dmt | Phe | Lys | NH₂ |
| D-Arg | Phe | Lys | Dmt | NH₂ |
| D-Arg | Phe | Dmt | Lys | NH₂ |
| D-Arg | Lys | Dmt | Phe | NH₂ |
| D-Arg | Lys | Phe | Dmt | NH₂ |
| Phe | Lys | Dmt | D-Arg | NH₂ |
| Phe | Lys | D-Arg | Dmt | NH₂ |
| Phe | D-Arg | Phe | Lys | NH₂ |
| Phe | D-Arg | Dmt | Lys | NH₂ |
| Phe | D-Arg | Lys | Dmt | NH₂ |
| Phe | Dmt | D-Arg | Lys | NH₂ |
| Phe | Dmt | Lys | D-Arg | NH₂ |
| Lys | Phe | D-Arg | Dmt | NH₂ |
| Lys | Phe | Dmt | D-Arg | NH₂ |
| Lys | Dmt | D-Arg | Phe | NH₂ |
| Lys | Dmt | Phe | D-Arg | NH₂ |
| Lys | D-Arg | Phe | Dmt | NH₂ |
| Lys | D-Arg | Dmt | Phe | NH₂ |
| D-Arg | Dmt | D-Arg | Phe | NH₂ |
| D-Arg | Dmt | D-Arg | Dmt | NH₂ |
| D-Arg | Dmt | D-Arg | Tyr | NH₂ |
| D-Arg | Dmt | D-Arg | Trp | NH₂ |
| Trp | D-Arg | Phe | Lys | NH₂ |
| Trp | D-Arg | Tyr | Lys | NH₂ |
| Trp | D-Arg | Trp | Lys | NH₂ |
| Trp | D-Arg | Dmt | Lys | NH₂ |
| D-Arg | Trp | Lys | Phe | NH₂ |
| D-Arg | Trp | Phe | Lys | NH₂ |
| D-Arg | Trp | Lys | Dmt | NH₂ |
| D-Arg | Trp | Dmt | Lys | NH₂ |
| D-Arg | Lys | Trp | Phe | NH₂ |
| D-Arg | Lys | Trp | Dmt | NH₂ |
| Cha | D-Arg | Phe | Lys | NH₂ |
| Ala | D-Arg | Phe | Lys | NH₂ |

Cha = cyclohexyl alanine

The amino acids of the peptides shown in Table 5 and 6 may be in either the L- or the D-configuration.

The peptides may be synthesized by any of the methods well known in the art. Suitable methods for chemically synthesizing the protein include, for example, those described by Stuart and Young in Solid Phase Peptide Synthesis, Second Edition, Pierce Chemical Company (1984), and in Methods Enzymol. 289, Academic Press, Inc, New York (1997).

### Prophylactic and Therapeutic Uses of Aromatic-Cationic Peptides.

The aromatic-cationic peptides described herein, such as D-Arg-2′,6′-Dmt-Lys-Phe-NH₂, or a pharmaceutically acceptable salt thereof, such as acetate or trifluoroacetate salt, are useful to prevent or treat disease. Specifically, the disclosure provides for both prophylactic and therapeutic methods of treating a subject suffering from, at risk of (or susceptible to) LHON. Accordingly, the present methods provide for the prevention and/or treatment of LHON in a subject by administering an effective amount of an aromatic-cationic peptide, such as D-Arg-2′,6′-Dmt-Lys-Phe-NH₂, or a pharmaceutically acceptable salt thereof, such as acetate or trifluoroacetate salt, to a subject in need thereof. For example, a subject can be administered an aromatic-cationic peptide compositions in an effort to improve one or more of the factors contributing to LHON.

One aspect of the technology includes methods of alleviating or eliminating the symptoms of LHON in a subject for therapeutic purposes. In therapeutic applications, compositions or medicaments are administered to a subject suspected of, or already suffering from such a disease in an amount sufficient to cure, or at least partially arrest, the symptoms of the disease, including its complications and intermediate pathological phenotypes in development of the disease. As such, the disclosure provides methods of treating an individual afflicted with LHON.

In one aspect, the invention provides a method for preventing LHON in a subject by administering to the subject an aromatic-cationic peptide such as D-Arg-2′,6′-Dmt-Lys-Phe-NH₂, or a pharmaceutically acceptable salt thereof, such as acetate or trifluoroacetate salt that modulates one or more signs or symptoms of LHON. Subjects at risk for LHON can be identified by, e.g., any or a combination of diagnostic or prognostic assays as described herein. In prophylactic applications, pharmaceutical compositions or medicaments of aromatic-cationic peptides such as D-Arg-2′,6′-Dmt-Lys-Phe-NH₂, or a pharmaceutically acceptable salt thereof, such as acetate or trifluoroacetate salt are administered to a subject susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. Administration of a prophylactic aromatic-cationic can occur prior to the manifestation of symptoms characteristic of the aberrancy, such that a disease or disorder is prevented or, alternatively, delayed in its progression. Depending upon the type of aberrancy, e.g., an aromatic-cationic peptide which acts to enhance or improve mitochondrial function or reduce oxidative damage can be used for treating the subject. The appropriate compound can be determined based on screening assays described herein.

Determination of the Biological Effect of the Aromatic-Cationic Peptide-Based Therapeutic. In various embodiments, suitable in vitro or in vivo assays are performed to determine the effect of a specific aromatic-cationic peptide-based therapeutic and whether its administration is indicated for treatment. In various embodiments, in vitro assays can be performed with representative cells of the type(s) involved in the subject's disorder, to determine if a given aromatic-cationic peptide-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In one embodiment, administration of an aromatic-cationic peptide to a subject exhibiting symptoms associated with LHON will cause an improvement in one or more of those symptoms.

### Modes of Administration and Effective Dosages

Any method known to those in the art for contacting a cell, organ or tissue with a peptide may be employed. Suitable methods include in vitro, ex vivo, or in vivo methods. In vivo methods typically include the administration of an aromatic-cationic peptide, such as those described above, to a mammal, such as a human. When used in vivo for therapy, the aromatic-cationic peptides are administered to the subject in effective amounts (i.e., amounts that have desired therapeutic effect). The dose and dosage regimen will depend upon the extent or severity of LHON in the subject, the characteristics of the particular aromatic-cationic peptide used, e.g., its therapeutic index, the subject, and the subject's history.

The peptides disclosed herein may be formulated as pharmaceutically acceptable salts. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the salt is an acetate salt. Additionally or alternatively, in other embodiments, the salt is a trifluoroacetate salt. In some embodiments, the salt is a tartrate salt.

The effective amount may be determined during pre-clinical trials and clinical trials by methods familiar to physicians and clinicians. An effective amount of a peptide useful in the methods of the present invention, such as in a pharmaceutical composition, may be administered to a mammal in need thereof by any of a number of well-known methods for administering pharmaceutical compounds. In some embodiments, the peptide may be administered systemically, topically, or intraocularly.

The aromatic-cationic peptides described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The aromatic-cationic peptide compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it may be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For ophthalmic applications, the therapeutic compound is formulated into solutions, suspensions, and ointments appropriate for use in the eye. For ophthalmic formulations generally, see Mitra (ed.), Ophthalmic Drug Delivery Systems, Marcel Dekker, Inc., New York, N.Y. (1993) and also Havener, W. H., Ocular Pharmacology, C.V. Mosby Co., St. Louis (1983). Ophthalmic pharmaceutical compositions may be adapted for topical administration to the eye in the form of solutions, suspensions, ointments, creams or as a solid insert. For a single dose, from between 0.1 ng to 5000 μg, 1 ng to 500 μg, or 10 ng to 100 μg of the aromatic-cationic peptides can be applied to the human eye.

The ophthalmic preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable level of the aromatic-cationic peptide in the eye. Administration to the mammalian eye may be about once or twice daily.

Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic protein or peptide can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34 (7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of crythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods 4 (3) 201-9 (1994); and Gregoriadis, “Engineering Liposomes for Drug Delivery: Progress and Problems,” Trends Biotechnol. 13 (12):527-37 (1995). Mizguchi et al., Cancer Lett. 100:63-69 (1996).

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies ideally within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the aromatic-cationic peptides, sufficient for achieving a therapeutic or prophylactic effect, range from about 0.000001 mg per kilogram body weight per day to about 10,000 mg per kilogram body weight per day. In some embodiments, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.1-10,000 micrograms per kg body weight. In one embodiment, aromatic-cationic peptide concentrations in a carrier range from 0.2 to 2000 micrograms per delivered milliliter. An exemplary treatment regime entails administration once per day or once a week. Intervals can also be irregular as indicated by measuring blood levels of glucose or insulin in the subject and adjusting dosage or administration accordingly. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of an aromatic-cationic peptide may be defined as a concentration of peptide at the target tissue of 10⁻¹¹ to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

In some embodiments, the dosage of the aromatic-cationic peptide is provided at a “low,” “mid,” or “high” dose level. In one embodiment, the low dose is provided from about 0.0001 to about 0.5 mg/kg/h, suitably from about 0.01 to about 0.1 mg/kg/h. In one embodiment, the mid-dose is provided from about 0.1 to about 1.0 mg/kg/h, suitably from about 0.1 to about 0.5 mg/kg/h. In one embodiment, the high dose is provided from about 0.5 to about 10 mg/kg/h, suitably from about 0.5 to about 2 mg/kg/h.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

Combination Therapy with an Aromatic-Cationic Peptide and Other Therapeutic Agents

In certain instances, it may be appropriate to administer at least one of the aromatic-cationic peptides described herein (or a pharmaceutically acceptable salt, ester, amide, prodrug, or solvate) in combination with another therapeutic agent. By way of example only, if one of the side effects experienced by a patient upon receiving one of the aromatic-cationic peptides herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit in the prevention or treatment of LHON. By way of example only, in a treatment for LHON involving administration of one of the aromatic-cationic peptides described herein, increased therapeutic benefit may result by also providing the patient with other therapeutic agents or therapies for LHON. In any case, the overall benefit experienced by the patient may simply be additive of the two therapeutic agents or the patient may experience a synergistic benefit.

A “synergistic effect” refers to a greater-than-additive therapeutic effect which is produced by a combination of at least two therapeutic agents, and which exceeds that which would otherwise result from administration of any individual therapeutic agent alone. Therefore, lower doses of one or more of the individual therapeutic agents may be used in treating LHON, e.g., disruptions in mitochondrial oxidative phosphorylation, resulting in increased therapeutic efficacy and decreased side-effects.

Specific, non-limiting examples of possible combination therapies include use of at least one aromatic-cationic peptide with nitric oxide (NO) inducers, statins, negatively charged phospholipids, antioxidants, minerals, anti-inflammatory agents, anti-angiogenic agents, matrix metalloproteinase inhibitors, and carotenoids. In several instances, suitable combination agents may fall within multiple categories (by way of example only, lutein is an antioxidant and a carotenoid). Further, the aromatic-cationic peptides may also be administered with additional agents that may provide benefit to the patient, including by way of example only cyclosporin A.

In addition, the aromatic-cationic peptides may also be used in combination with procedures that may provide additional or synergistic benefit to the patient, including, by way of example only, the use of extracorporeal rheopheresis (also known as membrane differential filtration), the use of implantable miniature telescopes, laser photocoagulation of drusen, and microstimulation therapy.

The use of antioxidants has been shown to benefit patients with ophthalmic disorders. See, e.g., Arch. Ophthalmol., 119: 1417-36 (2001); Sparrow, et al., J. Biol. Chem., 278:18207-13 (2003). Examples of suitable antioxidants that could be used in combination with at least one aromatic-cationic peptide include vitamin C, vitamin E, beta-carotene and other carotenoids, coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

The use of certain minerals has also been shown to benefit patients with ophthalmic disorders. See, e.g., Arch. Ophthalmol., 119: 1417-36 (2001). Examples of suitable minerals that could be used in combination with at least one aromatic-cationic peptide include copper-containing minerals, such as cupric oxide; zinc-containing minerals, such as zinc oxide; and selenium-containing compounds.

The use of certain negatively-charged phospholipids has also been shown to benefit patients with ophthalmic disorders. See, e.g., Shaban & Richter, Biol. Chem., 383:537-45 (2002); Shaban, et al., Exp. Eye Res., 75:99-108 (2002). Examples of suitable negatively charged phospholipids that could be used in combination with at least one aromatic-cationic peptide include cardiolipin and phosphatidylglycerol. Positively-charged and/or neutral phospholipids may also provide benefit for patients with o ophthalmic disorders when used in combination with aromatic-cationic peptides.

The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a very high number of apoptotic photoreceptor cells, while the group with high zeaxanthin concentrations had minimal damage. Examples of suitable carotenoids for in combination with at least one aromatic-cationic peptide include lutein and zeaxanthin, as well as any of the aforementioned carotenoids.

Suitable nitric oxide inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoargininc), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the endothelium, and has been identified as nitric oxide or a closely related derivative thereof (Palmer et al, Nature, 327:524-526 (1987); Ignarro et al, Proc. Natl. Acad. Sci. USA, 84:9265-9269 (1987)).

Statins serve as lipid-lowering agents and/or suitable nitric oxide inducers. In addition, a relationship has been demonstrated between statin use and delayed onset or development of certain ophthalmic disorders. G. McGwin, et al., British Journal of Ophthalmology, 87:1121-25 (2003). Statins can thus provide benefit to a patient suffering from LHON when administered in combination with aromatic-cationic peptides. Suitable statins include, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

Suitable anti-inflammatory agents with which the aromatic-cationic peptides may be used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, or Celebrex™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

Suitable matrix metalloproteinases (MMPs) inhibitors may also be administered in combination with aromatic-cationic peptides in order to treat LHON or symptoms associated with LHON. MMPs are known to hydrolyze most components of the extracellular matrix. These proteinases play a central role in many biological processes such as normal tissue remodeling, embryogenesis, wound healing and angiogenesis. However, excessive expression of MMP has been observed in many disease states, including certain ophthalmic disorders. Many MMPs have been identified, most of which are multidomain zinc endopeptidases. A number of metalloproteinase inhibitors are known (see for example the review of MMP inhibitors by Whittaker M. et al, Chemical Reviews 99(9):2735-2776 (1999)). Representative examples of MMP Inhibitors include Tissue Inhibitors of Metalloproteinases (TIMPs) (e.g., TIMP-1, TIMP-2, TIMP-3, or TIMP-4), α-2-macroglobulin, tetracyclines (e.g., tetracycline, minocycline, and doxycycline), hydroxamates (e.g., BATIMASTAT, MARIMISTAT and TROCADE), chelators (e.g., EDTA, cysteine, acetylcysteine, D-penicillamine, and gold salts), synthetic MMP fragments, succinyl mercaptopurines, phosphonamidates, and hydroxaminic acids. Examples of MMP inhibitors that may be used in combination with aromatic cationic peptides include, by way of example only, any of the aforementioned inhibitors.

The use of antiangiogenic or anti-VEGF drugs has also been shown to provide benefit for patients with ophthalmic disorders. Examples of suitable antiangiogenic or anti-VEGF drugs that could be used in combination with at least one aromatic-cationic peptide include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone—ru486), subtenon triamcinolone acetonide, intravitreal crystalline triamcinolone acetonide, Prinomastat (AG3340—synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis).

Other pharmaceutical therapies that have been used to relieve visual impairment can be used in combination with at least one aromatic-cationic peptide. Such treatments include but are not limited to agents such as Visudyne™ with use of a non-thermal laser, PKC 412, Endovion (NeuroScarch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), ranibizumab (Genentech), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group p1c), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporin A.

In any case, the multiple therapeutic agents may be administered in any order or even simultaneously. If simultaneously, the multiple therapeutic agents may be provided in a single, unified form, or in multiple forms (by way of example only, either as a single solution or as two separate solutions). One of the therapeutic agents may be given in multiple doses, or both may be given as multiple doses. If not simultaneous, the timing between the multiple doses may vary from more than zero weeks to less than about four weeks, less than about six weeks, less than about 2 months, less than about 4 months, less than about 6 months, or less than about one year. In addition, the combination methods, compositions and formulations are not to be limited to the use of only two agents. By way of example only, an aromatic-cationic peptide may be provided with at least one antioxidant and at least one negatively charged phospholipid; or an aromatic-cationic peptide may be provided with at least one antioxidant and at least one inducer of nitric oxide production; or an aromatic-cationic peptide may be provided with at least one inducer of nitric oxide productions and at least one negatively charged phospholipid; and so forth.

In addition, an aromatic-cationic peptide may also be used in combination with procedures that may provide additional or synergistic benefits to the patient. Procedures known, proposed or considered to relieve visual impairment include but are not limited to “limited retinal translocation”, photodynamic therapy (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

In some embodiments, aromatic-cationic peptides of the present technology are administered in combination with one or more agents used for the prophylaxis or treatment of LHON, including but not limited to, for example one or more of vitamins and/or nutritional supplements (including, but not limited to, for example, folic acid, vitamin B2, vitamin B12, vitamin C, and vitamin E), brimonidine, antioxidants, (including, but not limited to, for example, glutathione, Trolox (a derivative of vitamin E), curcumin, idebenone, and coenzyme Q-10), and cyclosporine A.

Further combinations that may be used to benefit an individual include using genetic testing to determine whether that individual is a carrier of a mutant gene that is known to be correlated with LHON. Patients possessing LHON-associated mutations are expected to find therapeutic and/or prophylactic benefit in the methods described herein.

### EXAMPLES

The present invention is further illustrated by the following examples, which should not be construed as limiting in any way.

### Example 1—Prevention of Leber's Hereditary Optic Neuropathy (LHON) in a Mammalian Subject

This example demonstrates the use of D-Arg-2′,6′-Dmt-Lys-Phe-NH₂ in the prevention of Leber's hereditary optic neuropathy (LHON). In particular, the example demonstrates the use of D-Arg-2′,6′-Dmt-Lys-Phe-NH₂ in preventing LHON in a mouse model of the disease.

Murine model. This example uses the murine model of LHON previously described by Lin, et al., Proc. Nat. Acad. Sci. 109(49):20065-20070 (2012). The animals harbor an ND6 P25L mutation. The LT13 cell line corresponds to the ND6 P25L mutant fibroblast line used for mouse embryonic stem cell fusions.

Mice harboring the ND6 P25L mutation are administered 1-10 μg D-Arg-2′,6′-Dmt-Lys-Phe-NH₂ or saline vehicle subcutaneously once daily from 0-14 months of age. In another treatment group, ND6 P25L mutant mice receive 1 drop of 1%, 3%, or 5% D-Arg-2′,6′-Dmt-Lys-Phe-NH₂ ophthalmic solution or saline vehicle in both eyes three times per day from 0-14 months of age. Various aspects of LHON are assessed in treatment and control animals at 14 and 24 months of age, with the ND6 P25L compared to wild-type mice for each parameter measured.

It is expected that administration of D-Arg-2′,6′-Dmt-Lys-Phe-NH₂ once daily from 0-14 months of age will prevent the onset of, delay the onset of, and/or reduce the severity (e.g., ameliorate) of the effects of the ND6 P25L mutation, thereby preventing or ameliorating LHON and its symptoms. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Reduced Retinal Response. The ND6 P25L mice are examined for ocular function by electroretinogram beginning at 14 months of age. It is expected that the animals will show a significant deficit in nearly all parameters examined. The scotopic B wave of dark-adapted ND6 P25L eyes is expected to be reduced in amplitude by approximately 25.5% and approximately 33.1% with 0.01 and 1 cd·s/m2 (maximum) stimulations. The scotopic A-wave of ND6 P25L mutant eyes is expected to show approximately a 23% reduction. The scotopic oscillatory potentials (OPs), a high-frequency response derived from multiple retinal cell types, are expected to show approximately a 20.7% and approximately a 21.7% reduction with 0.01 and 1 cd·s/m2 stimulations. Photopic B-wave ERG amplitude, measuring cone functions, is expected to be decreased approximately 17.7%. There is further expected a trend toward increased latencies to the A and B waves. Despite the functional deficit observed in the ERGs, it is expected that the ND6 P25L mutants will not exhibit reduced visual responses, as assessed by optokinetic analysis.

It is expected that administration of D-Arg-2′,6′-Dmt-Lys-Phe-NH₂ once daily from 0-14 months of age will prevent the onset of, delay the onset of, or reduce the severity of (e.g. ameliorate) these effects in ND6 P25L mutant animals, thereby preventing these aspects of LHON. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

RGC Axonal Swelling and Preferential Loss of Smallest Fibers. Electron microscopic analysis of RGC axons is expected to reveal that ND6 P25L mutants exhibit axonal swelling in the optic nerve. The average axonal diameter is expected to be approximately 0.67 m in wild-type and approximately 0.80 μm in ND6 P25L mutant 14-month-old mice, and approximately 0.73 m in wild-type and approximately 0.85 μm in ND6 P25L mutant mice at 24 months of age. Fourteen-month-old ND6 P25L mutant mice are expected to have an increased number of large fibers but fewer small axonal fibers (=0.5 μm). The change in axonal diameters is expected to be more pronounced in 24-month-old ND6 P25L mice. Hence, ND6 P25L mice are expected to have fewer small and medium axons (=0.8 μm) and more swollen axonal fibers with diameters larger than 1 μm. This effect is expected to be the most severe in the area of the smallest fibers in the central and temporal regions of the mouse optic nerve, which corresponds to the human temporal region most affected in LHON.

Quantification of the number of axons in the optic nerves is expected to reveal no significant difference in the total counts at 14 months of age, and approximately a 30% reduction at 24 months of age. Thus, the observed shift toward larger axons is predicted to be attributable initially (14 months) to swelling of medium axons, and later (24 months), to the loss of small axons.

It is expected that administration of D-Arg-2′,6′-Dmt-Lys-Phe-NH₂ once daily from 0-14 months of age will prevent the onset of, delay the onset of, or reduce the severity of these effects in ND6 P25L mutant animals, thereby preventing or ameliorating these aspects of LHON. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Abnormal Mitochondrial Morphology and Proliferation in RGC Axons. Mitochondria in the optic tracts of the ND6 P25L mutants are expected to be abnormal and increased in number, consistent with the compensatory mitochondrial proliferation observed in LHON patients. The optic tract axons of 14-month-old ND6 P25L mice are expected to have approximately a 58% increase in mitochondria, with 24-month-old animals having approximately a 94% increase. The ND6 P25L mitochondria are expected to appear hollowed with irregular cristae, with approximately 31.5% more of the ND6 P25L mitochondria being abnormal at 14 months and approximately 56% more at 24 months of age. Axons filled with abnormal mitochondria are expected to demonstrate marked thinning of the myelin sheath.

It is expected that administration of D-Arg-2′,6′-Dmt-Lys-Phe-NH₂ once daily from 0-14 months of age will prevent the onset of, delay the onset of, or reduce the severity of these effects in ND6 P25L mutant animals, thereby preventing or ameliorating these aspects of LHON. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Altered Liver Mitochondria Complex I Activity. The complex I activity of the ND6 P25L mice is assayed in liver mitochondria. Results are expected to demonstrate that rotenone-sensitive NADH:ubiquinone oxidoreductase activity is decreased by approximately 29%, which is equivalent to the reduction seen in the LT13 cell line. It is expected that the decrease in activity will not be attributable to a lower abundance of complex I, as it is expected that the NADH:ferricyanide oxidoreductase will be unaltered in the ND6 mutant mice. It is further expected that the ND6 mutation will cause approximately a 25% decrease in mitochondrial oxygen consumption, also seen in the LT13 cell line.

It is expected that administration of D-Arg-2′,6′-Dmt-Lys-Phe-NH₂ once daily from 0-14 months of age will prevent the onset of, delay the onset of, or reduce the severity of these effects in ND6 P25L mutant animals, thereby preventing or ameliorating these aspects of LHON. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Increased Forward Electron Flow but Lack of Reverse Electron Flow ROS Production in Liver Mitochondria. Analysis of mitochondrial H₂O₂ production in ND6 P25L mutant liver mitochondria is expected to show little difference in reactive oxygen species (ROS) production using site 1 substrates (glutamate and malate). However, when complex 1 H₂O₂ production is measured during reverse electron transfer (RET) driven by succinate in the presence of oligomycin, it is expected that ND6 P25L mutants will show an almost complete absence of ROS production.

Because the measurable rate of H₂O₂ production during forward electron transfer is expected to be lower than in the LT13 cells, submitochondrial particles (SMPs), which lack much of the H₂O₂-detoxification systems, will also be tested. It is expected that ND6 P25L mutant SMPs will show a significant increase in H₂O₂ production during forward electron transfer that is comparable to that seen in LT13 cells.

It is expected that administration of D-Arg-2′,6′-Dmt-Lys-Phe-NH₂ once daily from 0-14 months of age will prevent the onset of, delay the onset of, or reduce the severity of these effects in ND6 P25L mutant animals, thereby preventing or ameliorating these aspects of LHON. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Reduced Complex I Activity and Increased Forward but Not Reverse ROS Production in Synaptosomes. Mitochondrial function is examined in isolated synaptosomes, plasma membrane-bound synaptic boutons that encompass mitochondria and cytoplasmic biochemical machinery of the neuron. ND6 P25L mutant synaptosomes are expected to have reduced oxygen consumption under all conditions examined. The reduction is expected to be greatest under low-turnover conditions (basal or in the presence of oligomycin), with the deficit decreased as the mitochondrial membrane potential is reduced and respiration rate increased due to uncoupling with carbonyl cyanide-4-(trifluoromethoxy)phenylhydrazone (FCCP) or by activating the plasma membrane sodium ion channel with veratridine, thus increasing ATP consumption by the Na+-K+ ATPase. In contrast to cultured cell mitochondria in which ATP production is reduced, in situ ATP levels in the synaptosomes are expected to be maintained under various energetically demanding conditions, including incubation with 4-aminopyridine, a potassium ion channel inhibitor; a high concentration of KCl that depolarizes synaptosome; and veratridine. The ND6 P25L synaptosomes are further expected display a slight but significantly greater decrease in ATP levels compared to control mice when partially inhibited by rotenone (approximately 9.7% less than controls), similar to that previously reported for cytoplasmic hybrid cell lines harboring LHON mtDNA mutations.

Analysis of brain mitochondrial H₂O₂ production is expected to show that ND6 P25L mitochondria have elevated ROS production during forward complex I electron transport from glutamate and malate and marked suppression of ROS production during RET from succinate in the presence of oligomycin. Rotenone is expected to increase the rate of H₂O₂ production during forward electron transfer in both wild-type (approximately a 2.3-fold increase) and ND6 P25L (approximately a 2.7-fold increase) synaptosomes. Therefore, the ND6 P25L brain mitochondria are predicted to have an increased rate of H₂O₂ generation even where electron transfer to ubiquinone is fully inhibited by rotenone. During RET conditions, with succinate plus oligomycin, rotenone is expected to decrease the rate of generation of H₂O₂ in wild-type animals by approximately 79% and in ND6 P25L mice by approximately 59%. Under these conditions the rate of H₂O₂ generation is predicted to be equivalent for wild-type and ND6 P25L mice.

Synaptosomes respiring on glucose also are expected to show increased forward electron transfer H₂O₂ production, including in the presence of rotenone. These biochemical differences are expected to be kinetic in nature, with complex I subunit levels not decreased.

To determine whether the increased ROS production has cellular consequences, 3-nitrotyrosine and glial fibrillary acid protein (GFAP) levels is measured. It is expected that the 3-nitrotyrosine level will be increased more than two-fold in ND6 P25L mice compared to controls, with and GFAP increased approximately 65.5%.

It is expected that administration of D-Arg-2′,6′-Dmt-Lys-Phe-NH₂ once daily from 0-14 months of age will prevent the onset of, delay the onset of, or reduce the severity of these effects in ND6 P25L mutant animals, thereby preventing or ameliorating these aspects of LHON. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Results. These results will show that aromatic-cationic peptides of the present technology, such as D-Arg-2′,6′-Dmt-Lys-Phe-NH₂ are useful for preventing the onset of, delaying the onset of, and/or reducing the severity of the symptoms of LHON in a mammalian subject. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone. As such, aromatic-cationic peptides of the present technology, such as D-Arg-2′,6′-Dmt-Lys-Phe-NH₂ are useful in methods for preventing LHON and/or ameliorating the symptoms of LHON in a mammalian subject in need thereof comprising administering a therapeutically effective amount of an aromatic-cationic peptide, such as D-Arg-2′,6′-Dmt-Lys-Phe-NH₂.

### Example 2—Treatment of Leber's Hereditary Optic Neuropathy in a Mammalian Subject (LHON)

This example demonstrates the use of D-Arg-2′,6′-Dmt-Lys-Phe-NH₂ in the prevention of Leber's hereditary optic neuropathy (LHON). In particular, the example demonstrates the use of D-Arg-2′,6′-Dmt-Lys-Phe-NH₂ in preventing LHON in a mouse model of the disease.

Murine model. This example uses the murine model of LHON previously described by Lin, et al., Proc. Nat. Acad. Sci. 109(49):20065-20070 (2012). The animals harbor an ND6 P25L mutation. The LT13 cell line corresponds to the ND6 P25L mutant fibroblast line used for mouse embryonic stem cell fusions.

Mice harboring the ND6 P25L mutation are administered 1-10 g D-Arg-2′,6′-Dmt-Lys-Phe-NH₂ or saline vehicle subcutaneously once daily from 14-24 months of age, after the onset of LHON as established by the criteria set forth in Example 1. In another treatment group, ND6 P25L mutant mice receive 1 drop of 1%, 3%, or 5% D-Arg-2′,6′-Dmt-Lys-Phe-NH₂ ophthalmic solution or saline vehicle in both eyes three times per day from 14-24 months of age. Various aspects of LHON are assessed in treatment and control animals at 14 and 24 months of age, with the ND6 P25L compared to wild-type mice for each parameter measured.

It is expected that administration of D-Arg-2′,6′-Dmt-Lys-Phe-NH₂ once daily from 14-24 months of age will reduce or eliminate the effects of the ND6 P25L mutation, thereby treating LHON or ameliorating the symptoms of LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Reduced Retinal Response. The ND6 P25L mice are examined for ocular function by electroretinogram beginning at 14 months of age. It is expected that the animals will show a significant deficit in nearly all parameters examined. The scotopic B wave of dark-adapted ND6 P25L eyes is expected to be reduced in amplitude by approximately 25.5% and approximately 33.1% with 0.01 and 1 cd·s/m2 (maximum) stimulations. The scotopic A-wave of ND6 P25L mutant eyes is expected to show approximately a 23% reduction. The scotopic oscillatory potentials (OPs), a high-frequency response derived from multiple retinal cell types, are expected to show approximately a 20.7% and approximately a 21.7% reduction with 0.01 and 1 cd·s/m2 stimulations. Photopic B-wave ERG amplitude, measuring cone functions, is expected to be decreased approximately 17.7%. There is further expected a trend toward increased latencies to the A and B waves. Despite the functional deficit observed in the ERGs, it is expected that the ND6 P25L mutants will not exhibit reduced visual responses, as assessed by optokinetic analysis.

It is expected that administration of D-Arg-2′,6′-Dmt-Lys-Phe-NH₂ once daily from 14-24 months of age will reduce or eliminate the effects of the ND6 P25L mutation, thereby treating LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

RGC Axonal Swelling and Preferential Loss of Smallest Fibers. Electron microscopic analysis of RGC axons is expected to reveal that ND6 P25L mutants exhibit axonal swelling in the optic nerve. The average axonal diameter is expected to be approximately 0.67 m in wild-type and approximately 0.80 μm in ND6 P25L mutant 14-month-old mice, and approximately 0.73 m in wild-type and approximately 0.85 μm in ND6 P25L mutant mice at 24 months of age. Fourteen-month-old ND6 P25L mutant mice are expected to have an increased number of large fibers but fewer small axonal fibers (=0.5 μm). The change in axonal diameters is expected to be more pronounced in 24-month-old ND6 P25L mice. Hence, ND6 P25L mice are expected to have fewer small and medium axons (=0.8 μm) and more swollen axonal fibers with diameters larger than 1 μm. This effect is expected to be the most severe in the area of the smallest fibers in the central and temporal regions of the mouse optic nerve, which corresponds to the human temporal region most affected in LHON.

Quantification of the number of axons in the optic nerves is expected to reveal no significant difference in the total counts at 14 months of age, and approximately a 30% reduction at 24 months of age. Thus, the observed shift toward larger axons is predicted to be attributable initially (14 months) to swelling of medium axons, and later (24 months), to the loss of small axons.

It is expected that administration of D-Arg-2′,6′-Dmt-Lys-Phe-NH₂ once daily from 14-24 months of age will reduce or eliminate the effects of the ND6 P25L mutation, thereby treating LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Abnormal Mitochondrial Morphology and Proliferation in RGC Axons. Mitochondria in the optic tracts of the ND6 P25L mutants are expected to be abnormal and increased in number, consistent with the compensatory mitochondrial proliferation observed in LHON patients. The optic tract axons of 14-month-old ND6 P25L mice are expected to have approximately a 58% increase in mitochondria, with 24-month-old animals having approximately a 94% increase. The ND6 P25L mitochondria are expected to appear hollowed with irregular cristae, with approximately 31.5% more of the ND6 P25L mitochondria being abnormal at 14 months and approximately 56% more at 24 months of age. Axons filled with abnormal mitochondria are expected to demonstrate marked thinning of the myelin sheath.

It is expected that administration of D-Arg-2′,6′-Dmt-Lys-Phe-NH₂ once daily from 14-24 months of age will reduce or eliminate the effects of the ND6 P25L mutation, thereby treating LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Altered Liver Mitochondria Complex I Activity. The complex I activity of the ND6 P25L mice is assayed in liver mitochondria. Results are expected to demonstrate that rotenone-sensitive NADH:ubiquinone oxidoreductase activity is decreased by approximately 29%, which is equivalent to the reduction seen in the LT13 cell line. It is expected that the decrease in activity will not be attributable to a lower abundance of complex I, as it is expected that the NADH:ferricyanide oxidoreductase will be unaltered in the ND6 mutant mice. It is further expected that the ND6 mutation will cause approximately a 25% decrease in mitochondrial oxygen consumption, also seen in the LT13 cell line.

It is expected that administration of D-Arg-2′,6′-Dmt-Lys-Phe-NH₂ once daily from 14-24 months of age will reduce or eliminate the effects of the ND6 P25L mutation, thereby treating LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Increased Forward Electron Flow but Lack of Reverse Electron Flow ROS Production in Liver Mitochondria. Analysis of mitochondrial H₂O₂ production in ND6 P25L mutant liver mitochondria is expected to show little difference in reactive oxygen species (ROS) production using site 1 substrates (glutamate and malate). However, when complex I H₂O₂ production is measured during reverse electron transfer (RET) driven by succinate in the presence of oligomycin, it is expected that ND6 P25L mutants will show an almost complete absence of ROS production.

Because the measurable rate of H₂O₂ production during forward electron transfer is expected to be lower than in the LT13 cells, submitochondrial particles (SMPs), which lack much of the H₂O₂-detoxification systems, will also be tested. It is expected that ND6 P25L mutant SMPs will show a significant increase in H₂O₂ production during forward electron transfer that is comparable to that seen in LT13 cells.

It is expected that administration of D-Arg-2′,6′-Dmt-Lys-Phe-NH₂ once daily from 14-24 months of age will reduce or eliminate the effects of the ND6 P25L mutation, thereby treating LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Reduced Complex I Activity and Increased Forward but Not Reverse ROS Production in Synaptosomes. Mitochondrial function is examined in isolated synaptosomes, plasma membrane-bound synaptic boutons that encompass mitochondria and cytoplasmic biochemical machinery of the neuron. ND6 P25L mutant synaptosomes are expected to have reduced oxygen consumption under all conditions examined. The reduction is expected to be greatest under low-turnover conditions (basal or in the presence of oligomycin), with the deficit decreased as the mitochondrial membrane potential is reduced and respiration rate increased due to uncoupling with carbonyl cyanide-4-(trifluoromethoxy)phenylhydrazone (FCCP) or by activating the plasma membrane sodium ion channel with veratridine, thus increasing ATP consumption by the Na+-K+ ATPase. In contrast to cultured cell mitochondria in which ATP production is reduced, in situ ATP levels in the synaptosomes are expected to be maintained under various energetically demanding conditions, including incubation with 4-aminopyridine, a potassium ion channel inhibitor; a high concentration of KCl that depolarizes synaptosome; and veratridine. The ND6 P25L synaptosomes are further expected display a slight but significantly greater decrease in ATP levels compared to control mice when partially inhibited by rotenone (approximately 9.7% less than controls), similar to that previously reported for cytoplasmic hybrid cell lines harboring LHON mtDNA mutations.

Analysis of brain mitochondrial H₂O₂ production is expected to show that ND6 P25L mitochondria have elevated ROS production during forward complex I electron transport from glutamate and malate and marked suppression of ROS production during RET from succinate in the presence of oligomycin. Rotenone is expected to increase the rate of H₂O₂ production during forward electron transfer in both wild-type (approximately a 2.3-fold increase) and ND6 P25L (approximately a 2.7-fold increase) synaptosomes. Therefore, the ND6 P25L brain mitochondria are predicted to have an increased rate of H₂O₂ generation even where electron transfer to ubiquinone is fully inhibited by rotenone. During RET conditions, with succinate plus oligomycin, rotenone is expected to decrease the rate of generation of H₂O₂ in wild-type animals by approximately 79% and in ND6 P25L mice by approximately 59%. Under these conditions the rate of H₂O₂ generation is predicted to be equivalent for wild-type and ND6 P25L mice.

Synaptosomes respiring on glucose also are expected to show increased forward electron transfer H₂O₂ production, including in the presence of rotenone. These biochemical differences are expected to be kinetic in nature, with complex I subunit levels not decreased.

To determine whether the increased ROS production has cellular consequences, 3-nitrotyrosine and glial fibrillary acid protein (GFAP) levels will be measured. It is expected that the 3-nitrotyrosine level will be increased more than two-fold in ND6 P25L mice compared to controls, with and GFAP increased approximately 65.5%.

It is expected that administration of D-Arg-2′,6′-Dmt-Lys-Phe-NH₂ once daily from 14-24 months of age will reduce or eliminate these effects of the ND6 P25L mutation, thereby treating LHON in ND6 P25L mutant mice. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone.

Results. These results will show that aromatic-cationic peptides of the present technology, such as D-Arg-2′,6′-Dmt-Lys-Phe-NH₂ are useful for reducing or eliminating the symptoms of LHON in a mammalian subject. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone. As such, aromatic-cationic peptides of the present technology, such as D-Arg-2′,6′-Dmt-Lys-Phe-NH₂ are useful in methods for treating LHON in a mammalian subject in need thereof comprising administering a therapeutically effective amount of an aromatic-cationic peptide, such as D-Arg-2′,6′-Dmt-Lys-Phe-NH₂.

### Example 3—Prevention and Treatment of Leber's Hereditary Optic Neuropathy in a Human Subject (LHON)

This example demonstrates the use of D-Arg-2′,6′-Dmt-Lys-Phe-NH₂ in the prevention and treatment of Leber's hereditary optic neuropathy (LHON). In particular, the example demonstrates the use of D-Arg-2′,6′-Dmt-Lys-Phe-NH₂ in preventing and treating LHON in a human subject.

Human subjects at risk of having, suspected of having, or diagnosed as having LHON are administered a therapeutically effective amount of an aromatic-cationic peptide of the present technology, such as D-Arg-2′,6′-Dmt-Lys-Phe-NH₂, alone or in conjunction with one or more additional therapeutic agents, on a therapeutic schedule appropriate for the prophylactic/therapeutic needs of the individual.

LHON patients are administered 1-10 μg D-Arg-2′,6′-Dmt-Lys-Phe-NH₂ or saline vehicle subcutaneously once daily for 12 months. In another treatment group, LHON patients receive 1 drop of 1%, 3%, or 5% D-Arg-2′,6′-Dmt-Lys-Phe-NH₂ ophthalmic solution or saline vehicle in both eyes three times per day for 12 months. Subjects are assessed periodically for signs and symptoms associated with LHON according to one or more criteria described herein.

It is expected that administration of an aromatic-cationic peptide of the present technology, such as D-Arg-2′,6′-Dmt-Lys-Phe-NH₂ to human subjects at risk of having, suspected of having, or diagnosed as having LHON will prevent the onset of, delay the onset of, or reduce the severity of the symptoms of LHON, thereby treating LHON in the subject. It is further expected that administration of D-Arg-2′,6′-Dmt-Lys-Phe-NH₂ in combination with one or more additional therapeutic agents will have synergistic effects in this regard compared to that resulting from administration of any individual therapeutic agent alone. These results will show that aromatic-cationic peptides of the present technology, such as D-Arg-2′,6′-Dmt-Lys-Phe-NH₂ are useful in methods for treating LHON in a human subject in need thereof comprising administering a therapeutically effective amount of an aromatic-cationic peptide, such as D-Arg-2′,6′-Dmt-Lys-Phe-NH₂.

### Example 4—Increase of ATP Synthesis and Cellular Respiration in Leber's Hereditary Optic Neuropathy (LHON) Cybrids

This example demonstrates the use of D-Arg-2′,6′-Dmt-Lys-Phe-NH₂ in the treatment of LHON. In particular, the example demonstrates that D-Arg-2′,6′-Dmt-Lys-Phe-NH₂ increases ATP synthesis and cellular respiration in LHON cybrids.

Methods and Materials

A cybrid (or cytoplasmic hybrid) is a eukaryotic cell line produced by the fusion of a whole cell with a cytoplast of another cell. Cytoplasts are derived from enucleated cells.

A particular method of cybrid formation involves the use of rho-zero (rho⁰) cells as the whole cell partner in the fusion. Rho-zero cells are cells that are depleted of their own mitochondrial DNA (mtDNA). Fusion of the cytoplast to the rho⁰ cell allows for the study of the mtDNA of the cytoplast in a “neutral” nuclear background, i.e., dissociate the genetic contribution of the mitochondrial genome from that of the nuclear genome.

### Method for Making Cybrid Cells

Cybrid cell lines are constructed using enucleated fibroblasts from a control individual (i.e., negative for LHON) and 3 unrelated probands with LHON as mitochondria donors. One control cybrid cell line and three LHON cybrid cell lines, one for each of the most common pathogenic mutations found in LHON, i.e., mutations at positions 11778/ND4, 3460/ND1, and 14484/ND6 of mtDNA, are produced.

The mitochondrial donor cytoplast is fused with the osteosarcoma (143B.TK-)-derived 206 cell line (rho⁰ 206 cell line). All fibroblast cell lines are established from skin biopsy samples or from umbilical cord specimens after having obtained the informed consent of LHON and control patients. Cell fusions of fibroblast-derived cytoplasts (enucleated fibroblasts) with the rho⁰ 206 cells are performed using the protocol, e.g., described in King et al., Science, 246: 500-03 (1989).

Parental and cybrid cell lines are grown in Dulbecco Modified Eagle Medium supplemented with 10% fetal bovine serum, 2 mM levoglutamine, penicillin G sodium (100 U/mL), streptomycin sulfate (100 μg/mL), and bromodeoxyuridine (0.1 mg/mL).

Method for measuring ATP synthesis

1 ml of each cybrid cell line at 5×10⁶ cells/ml is individually plated in tissue culture plates with growth media. The control cybrid cell line and each LHON cybrid cell line (i.e., 11778/ND4 cybrid, 3460/ND1 cybrid, and 14484/ND6 cybrid) are divided into treated and untreated groups, wherein the treated group is incubated with D-Arg-2′,6′-Dmt-Lys-Phe-NH₂ and the untreated group is incubated with phosphate-buffer solution (PBS). After incubation for 1 hour at 37° C., the ATP synthesis rate and cellular respiration are measured.

The ATP synthesis rate is assayed by permeabilizing the cells with digitonin according to the method described by Ouhabi et al., Anal Biochem., 263: 169-175 (1998). After permeabilization of the cells, 10 mM glutamate-10 mM malate (plus 0.6 mM malonate) and 0.5 mM adenosine diphosphate (ADP) are added to cells. The cells are incubated for 5 minutes at 30° C., and the reaction is terminated by adding 80% (vol/vol) dimethylsulfoxide. The ATP content is measured using the lucifcrin-lucifcrasc chemiluminescent method, e.g., described in Stanley et al., Anal Biochem., 29: 381-392 (1969).

Respiratory rates of digitonin-permeabilized cell samples are measured at 30° C. using a Clark-type oxygen electrode as previously described by Aicardi et al., Biochem Pharmacol., 31:3703-3705 (1982). The respiratory control ratio (RCR) is evaluated using glutamate-malate as a substrate.

Results

It is anticipated that LHON cybrid cells treated with 1-10 μg D-Arg-2′,6′-Dmt-Lys-Phe-NH₂ will display a greater ATP synthesis rate and have a higher respiratory rate as compared to the untreated LHON cybrid cells. These results will show that aromatic-cationic peptides of the present technology, such as D-Arg-2′,6′-Dmt-Lys-Phe-NH₂, are useful in methods for treating LHON.

### Example 5—Use of D-Arg-2′,6′-Dmt-Lys-Phe-NH₂ Ophthalmic Solution to Treat LHON Patients

This Example demonstrates the efficacy of D-Arg-2′,6′-Dmt-Lys-Phe-NH₂ ophthalmic solution in treating, ameliorating, or halting the progression of LHON in human subjects.

Approximately 70 male and female subjects with LHON of the genetic subtype m.11778G>A and loss of vision in both eyes of ≥1 year but ≤10 years duration will be recruited for a prospective, randomized, double-masked, vehicle controlled, multi-center study. Written informed consent will be obtained from all subjects or their legal guardians prior to screening.

### Patient Screening

LHON diagnosis will be based on clinical and ophthalmic functional/anatomic test findings and satisfactory documentation of the mitochondrial DNA genotype m.11778G>A. If a mitochondrial genotype has not been determined using reliable testing methods, the patient's status for the m.11778G>A genotype will be confirmed via mitochondrial DNA analysis. Once confirmed, data will be collected from a complete pre-treatment examination, consisting of vital signs, physical exam, urine pregnancy test for women of child-bearing potential, routine blood chemistries and urinalysis, measurement of best-corrected visual acuity (BCVA) using the ETDRS scale, manifest refraction, intraocular pressure (IOP) measurement, slit lamp examination and fundoscopy, fundus photography, evaluation of color discrimination and contrast sensitivity, Humphrey automated visual field testing (SITA FAST 30-2; both stimulus III and stimulus V), retinal nerve fiber layer thickness as measured by spectral domain optical coherence tomography (SD-OCT; Cirrus), photopic negative response electroretinography (PhNR-ERG), and the VFQ 39 visual quality-of-life questionnaire. This Screening examination will be performed no more than 30 days prior to the Baseline visit and may be combined with the Baseline visit. If applicable, urine pregnancy testing will be performed prior to initiation of treatment.

### Patient Selection

Inclusion criteria for the study are: (1) m.11778G>A mitochondrial DNA genotype, (2) ≥14 years of age, (3) BCVA of ≥CF 3′ equivalent in both eyes (OU), (4) Mean retinal nerve fiber thickness of between 60 microns to 80 microns OU (as measured by SD-OCT), (5) Media clarity, pupillary dilation, and patient cooperation sufficient for adequate ophthalmic visual function testing and anatomic assessment, (6) Ability to self-administer the ophthalmic solution as demonstrated at screening or having a care provider who can do so, and (7) Loss of vision in both eyes with clinically stable visual function (as assessed by the investigator) of ≥1 year but ≤10 years. Additionally, females of childbearing potential must agree to use one of the following methods of birth control from the date they sign the informed consent form until the conclusion of the study: (a) Abstinence, when it is in line with the preferred and usual lifestyle of the subject; (b) Maintenance of a monogamous relationship with a male partner who has been surgically sterilized by vasectomy (vasectomy procedure must have been conducted at least 60 days prior to the Screening Visit or confirmed via sperm analysis), (c) Barrier method (e.g. condom or occlusive cap) with spermicidal foam/gel/film/cream and either hormonal contraception (oral, implanted or injectable) or an intrauterine device or system.

Exclusion criteria include anyone or more of the following conditions:
Mean Deviation (MD) of <−30 dB on Humphrey automated visual field testing (SITA FAST 30-2, stimulus III);
Ocular hypertension or glaucoma, dry eye and any other ocular pathology requiring treatment with topical ophthalmic drops;
Cup to disc ratio of <0.8 in either eye;
Aphakia or intraocular lens placement in the anterior chamber of the study eye;
Any active ocular or peri-ocular infection or any history of recurrent or chronic infection or inflammation in the study eye;
History of herpetic infection in either eye;
History of corneal disease or surgery;
Current use or likely need for the use of contact lenses at any time during the study;
Concurrent disease in either the study eye or fellow control eye that could require medical or surgical intervention during the study period;
Media opacity, suboptimal pupillary dilatation, or refractive error that interferes with adequate retinal imaging;
History of allergic reaction to the investigational drug or any of its components;
Current use of or likely need for any excluded medication, including systemic medications known to be toxic to the lens, retina or optic nerve (e.g., deferoxamine, chloroquine/hydroxychloroquine (Plaquenil), tamoxifen, phenothiazines, ethambutol, and aminoglycosides);
Subjects that are immunocompromised or receiving immunosuppression therapy;
Any systemic or non-ocular symptoms that may be related to LHON;
Pregnant or lactating women;
Any disease or medical condition that in the opinion of the investigator would prevent the subject from participating in the study or might confound study results;
Participation in other investigational drug or device clinical trials within 30 days prior to enrollment, or planning to participate in any other investigational drug or device clinical trials within 30 days of study completion; and
Subjects unwilling or unable to comply with scheduled visits/examinations as described herein.

### Study Design

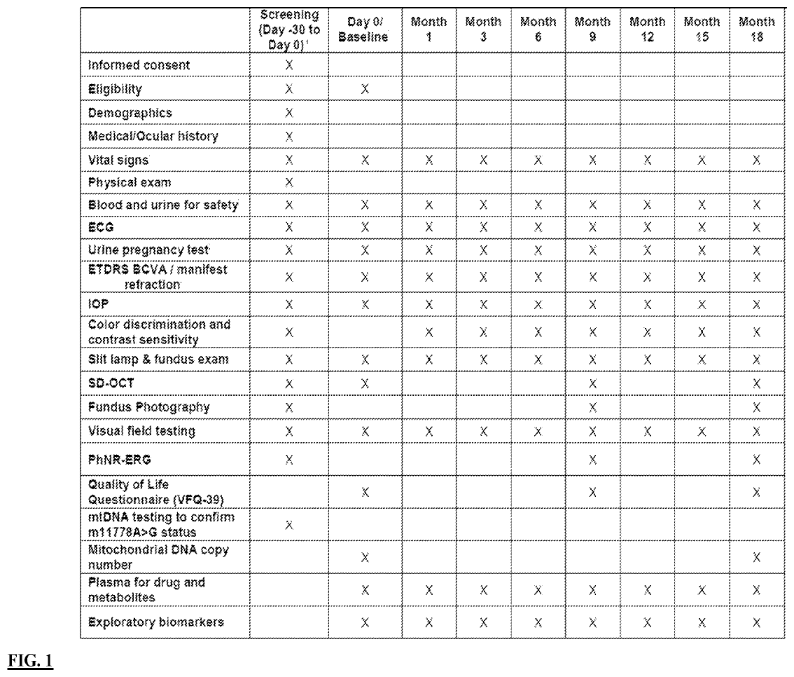

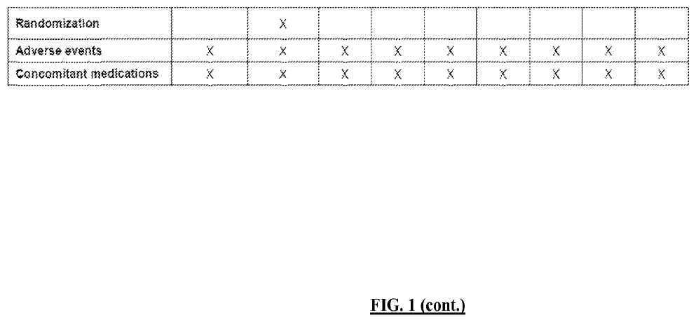

Patients that satisfy the above criteria will be randomized into experimental and control groups. Patients in the experimental group will receive 1 drop of 1%, 3%, or 5% D-Arg-2′,6′-Dmt-Lys-Phe-NH₂ ophthalmic solution in a randomly selected study eye three times per day for 18 months. The remaining eye of these patients serves as the untreated internal control. By contrast, the patients in the control group will be administered 1 drop of vehicle solution in one of their eyes (fellow control eye) three times per day over the course of the 18 month study. The schedule of clinical parameters to be determined at each patient visit is shown in FIG. 1. Plasma samples will be analyzed for the presence of D-Arg-2′,6′-Dmt-Lys-Phe-NH₂ and/or metabolites. Serum samples will be obtained in order to measure neuron specific enolase and to conduct phosphorylated axonal neurofilament analysis. As shown in FIG. 1, mitochondrial DNA copy number will be analyzed at Day 0 (Baseline) and at Month 18.

The therapeutic effect of D-Arg-2′,6′-Dmt-Lys-Phe-NH₂ will be assessed by measuring changes in visual field MD (both stimulus III and stimulus V), color discrimination/contrast sensitivity, BCVA, retinal nerve fiber layer thickness, VFQ-39 scores and PhNR-ERG response patterns at the different time points indicated in FIG. 1 compared to their corresponding Baseline values. Paired differences in change from Baseline in visual field MD in the study eye vs. fellow control eye will be analyzed using a paired T-test. The other efficacy parameters will be analyzed in a similar fashion.

Continuous variables will be summarized by descriptive statistics (sample size, mean, standard deviation, median, minimum and maximum). Discrete variables will be summarized by frequencies and percentages. Adverse events will be summarized by presenting the number and percentage of patients having any adverse event. Any other information collected (such as severity or relationship to study drug) will be listed as appropriate. In addition, a blinded interim analysis of data will be performed once approximately half of the subjects have completed twelve (12) months of treatment in order to assess the assumptions regarding variability. The sample size assumptions will be reviewed, and the number of planned subjects may be changed based on the blinded results.

Results

It is anticipated that the study eye of patients treated with D-Arg-2′,6′-Dmt-Lys-Phe-NH₂ ophthalmic solution will show improvements in at least one of the assessed clinical parameters of LHON (i.e., visual field MD, color discrimination/contrast sensitivity, BCVA, retinal nerve fiber layer thickness, VFQ-39 scores and PhNR-ERG response patterns) compared to the vehicle treated eyes of the control group. It is also anticipated that the rate of vision loss in the study eye of the treated subjects will be reduced compared to that observed in their untreated eye (internal control). These results will show that aromatic-cationic peptides of the present technology, such as D-Arg-2′,6′-Dmt-Lys-Phe-NH₂, are useful in methods for treating, ameliorating, or halting the progression of LHON in human subjects.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for treating or preventing Leber's hereditary optic neuropathy (LHON) in a mammalian subject in need thereof, comprising administering to the subject a therapeutically effective amount of a peptide represented by the formula D-Arg-2′,6′-Dmt-Lys-Phe-NH₂.

2. The method of claim 1, wherein the subject is a human.

3. The method of claim 1, wherein the peptide is administered intraocularly, iontophoretically, orally, topically, systemically, intravenously, subcutaneously, or intramuscularly.

4. The method of claim 1 further comprising separately, sequentially, or simultaneously administering a second active agent.

5. The method of claim 4, wherein the second active agent is selected from the group consisting of: a vitamin, an antioxidant, a metal complexer, an anti-inflammatory drug, an antibiotic, and an antihistamine.

6. The method of claim 5, wherein the antioxidant is vitamin A, vitamin C, vitamin E, lycopene, selenium, α-lipoic acid, coenzyme Q, glutathione, curcumin, idebenone, or a carotenoid.

7. The method of claim 4, wherein the second active agent is selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

8. The method of claim 5, wherein the vitamin is selected from the group consisting of: vitamin B2 and vitamin B12.
